(19) United States
(12) Patent Application Publication
(10) Pub. No.: US 2020/0392461 A1
(43) Pub. Date: Dec. 17, 2020

(54) IN VITRO AVIAN FOOD PRODUCT

(71) Applicant: JUST, INC., San Francisco, CA (US)

(72) Inventors: Nicholas MULLEN, San Francisco, CA (US); Nathaniel PARK, Alameda, CA (US); Christopher JONES, San Francisco, CA (US); Thomas BOWMAN, San Francisco, CA (US); Paola BIGNONE, Alameda, CA (US); Vitor Espirito SANTO, San Francisco, CA (US); Pavan KAMBAM, Sunnyvale, CA (US); Amranul HAQUE, Albany, CA (US); Ifeanyi Michael AMADI, Hayward, CA (US)

(21) Appl. No.: 16/900,719

(22) Filed: Jun. 12, 2020

Related U.S. Application Data
(60) Provisional application No. 62/861,948, filed on Jun. 14, 2019.

(51) Int. Cl. C12N 5/077; A23J 3/32; A23J 3/14; A23L 13/50

(52) U.S. Cl. CPC C12N 5/0656; A23J 3/32; A23J 3/14; A23L 13/50; C12N 2501/33; A23V 2002/00; C12N 2501/11; C12N 2501/998; C12N 2500/10; C12N 2501/999; C12N 2500/34; C12N 2501/115

(57) ABSTRACT

Provided herein are food products made in vitro from avian fibroblast cells and methods for harvesting the avian fibroblast cells. Particularly, an in vitro produced chicken product is produced. Also provided herein are methods of their production.

### FIELD

The present disclosure relates to food products derived from avian cells produced in vitro and methods of cultivation of avian cells in low serum or the absence of serum.

### BACKGROUND

Chicken has been a part of the human diet for thousands of years. The modern domestic chicken (Gallus domesticus) is descended from the red junglefowl (Gallus gallus), which is native to southeast Asia, though some related species may also have interbred in the evolution of the domestic chicken (Lawler et al.). It is believed to have been first domesticated in India around 2000 BCE (USDA Fact Sheet). Currently, there are believed to be about 2 billion chickens in the world, and they are poised to overtake pigs as the most common source of animal protein in the human diet (Gorman et al.). Because it has a high protein content and low fat content, chicken is a highly desirable food ingredient.

Chicken is a ubiquitous food of our era, crossing multiple cultural boundaries with ease. With its mild taste and uniform texture, chicken presents an intriguingly blank canvas for the flavor palette of almost any cuisine.

Chicken is often recommended as a healthier alternative to red meat. Chicken consumption is associated with a lower risk of colorectal cancer than red meat or processed meat (English et al.), and consumption of white meat (chicken, turkey and fish) is associated with lower risk of all-cause mortality, cancer risk, and cardiovascular disease (Sinha et al.). Also, chicken contains lower amounts of saturated fat and cholesterol, which are risk factors for cardiovascular disease, than red meat (International Agency for Research on Cancer).

Additionally, where safety concerns have arisen regarding chicken consumption, they typically include microbial contamination related to deficiencies in animal husbandry, slaughter, or processing practices, combined with undercooking that does not kill all of the microbes that may be on the chicken. During slaughter and processing, contamination of the meat with fecal matter is common. In random surveys of chicken products across the United States in 2012, the Physicians Committee for Responsible Medicine found 48% of samples to contain fecal matter, and a 2009 USDA study found that 87% of chicken carcasses tested positive for generic E. coli, a sign of fecal contamination, just prior to packaging. While thorough cooking can kill contaminating microorganisms, if cooking is not thorough, some microorganisms may survive to cause foodborne illness.

Cultured meat products have the potential to: (1) substantially reduce reliance on slaughtered animals for food use, (2) lessen the environmental burden of raising animals for food supply, and (3) provide a reliable source of protein that is both safe and has consistent quality.

### SUMMARY

The present disclosure provides methods for culturing avian fibroblast cells in vitro. The present disclosure also provides compositions for avian food products. This disclosure also sets forth processes for making and using products.

In some embodiments, there are provided methods of producing a food product comprising avian fibroblast cells cultured in vitro, the methods comprising culturing a population of avian fibroblast cells in vitro in a growth medium capable of maintaining the avian fibroblast cells, recovering the avian fibroblast cells, and formulating the recovered avian fibroblast cells into an edible food product. In some embodiments, the avian fibroblast cells comprise primary avian fibroblast cells. In some embodiments, the avian fibroblast cells comprise secondary avian fibroblast cells.

In some embodiments, there are provided methods of preparing a food product made from avian fibroblast cells grown in vitro, the method comprising the steps of: conditioning water with a phosphate to prepare conditioned water, hydrating a plant protein isolate or plant protein concentrate with the conditioned water to produce hydrated plant protein, contacting the cell paste with the hydrated plant protein to produce a cell and pulse protein mixture, heating the cell and plant protein mixture in steps, wherein the steps comprise at least one of:

ramping up the temperature of the cell and protein mixture to a temperature between 40-65° C., maintaining the temperature of the cell and protein mixture at a temperature between 40-65° C. for 1 to 30 minutes, ramping up the temperature of the cell and protein mixture to a temperature between 60-85° C., cooling the cell and protein mixture to a temperature between −1-25° C., and admixing the cell and protein mixture with a fat to create a pre-cooking product. The pre-cooking product can be consumed without further cooking. Alternatively, the pre-cooking product is cooked to produce the edible food product. Optionally, the pre-cooking product may be stored at room temperature, refrigeration temperatures or frozen.

In some embodiments, there are provided food products produced from avian fibroblasts, comprising a cell paste, the cell paste content of at least 5% by weight, and wherein the cell paste is made from avian fibroblast cells grown in vitro; a plant protein isolate or plant protein concentrate, the plant protein content at least 5% by weight; a fat, the fat content at least 5% by weight; and water, the water content at least 5% by weight.

In some embodiments, the food composition or food product comprises about 1%-100% by weight wet cell paste.

In some embodiments, plant protein isolates or plant protein concentrates are obtained from pulses selected from the group consisting of dry beans, lentils, mung beans, faba beans, dry peas, chickpeas, cowpeas, bambara beans, pigeon peas, lupins, vetches, adzuki, common beans, fenugreek, long beans, lima beans, runner beans, or tepary beans, soy beans, or mucuna beans. In various embodiments, the pulse protein isolates or plant protein concentrates provided herein are derived from Vigna angularis, Vicia faba, Cicer arietinum, Lens culinaris, Phaseolus vulgaris, Vigna unguiculata, Vigna subterranea, Cajanus cajan, Lupinus sp., Vetch sp., Trigonella foenum-graecum, Phaseolus lunatus, Phaseolus coccineus, or Phaseolus acutifolius. In some embodiments, the pulse protein isolates are derived from mung beans. In some embodiments, the mung bean is Vigna radiata.

In some embodiments, animal protein isolate and animal protein concentrate are obtained from animals or animal products. Examples of animal protein isolate or animal protein concentrate include whey, casein, and egg protein.

In some embodiments, plant protein isolates are obtained from wheat, rice, teff, oat, corn, barley, sorghum, rye, millet, triticale, amaranth, buckwheat, quinoa, almond, cashew, pecan, peanut, walnut, macadamia, hazelnut, pistachio, brazil, chestnut, kola nut, sunflower seeds, pumpkin seeds, flax seeds, cacao, pine nut, ginkgo, and other nuts.

### BRIEF DESCRIPTIONS OF THE DRAWINGS

FIG. 1 depicts a process diagram for culturing of avian fibroblast cells.

FIG. 2 depicts a process diagram for harvesting cultured avian fibroblast cells.

FIG. 3 depicts a hierarchical clustering of the transcriptome analysis of three biological replicates of chicken cell pools (JUST1, JUST2, JUST3) used to manufacture a cultured chicken meat product (JUST7, JUSTE, JUST9).

FIG. 4A depicts chicken fibroblast cell adaptation in low serum media indicating cell viability as a function of culture time. FIG. 4B depicts chicken fibroblast cell adaptation in low serum media indicating population doubling time as a function of passage number.

FIG. 5A depicts chicken fibroblast cell adaptation in basal media supplemented with fatty acids and growth factors as a function of culture time. FIG. 5B depicts chicken fibroblast cell adaptation in basal media without growth factors as a function of culture time. FIG. 5C depicts chicken fibroblast cell adaptation in serum free basal media supplemented with growth factors as a function of culture time. The growth factors comprise insulin-like, epidermal-like, and fibroblast-like growth factors.

FIG. 6A depicts the adaption of C1F chicken cells in media with decreasing concentrations of FBS in the presence of ITSEEF as defined herein, as a function of culture time. FIG. 6B depicts chicken fibroblast cell adaptation to serum-free media indicating the population doubling time as a function of passage number. FIG. 6C depicts cell viability as a function of time for the cultures shown in FIGS. 6A and 6B.

### DETAILED DESCRIPTION

The following description is presented to enable one of ordinary skill in the art to make and use the disclosed subject matter and to incorporate it in the context of applications. Various modifications, as well as a variety of uses in different applications, will be readily apparent to those skilled in the art, and the general principles defined herein may be applied to a wide range of embodiments. Thus, the present disclosure is not intended to be limited to the embodiments presented but is to be accorded the widest scope consistent with the principles and novel features disclosed herein.

### Definitions

As used herein, the term “batch culture” refers to a closed culture system with nutrient, temperature, pressure, aeration, and other environmental conditions to optimize growth. Because nutrients are not added, nor waste products removed during incubation, batch cultures can complete a finite number of life cycles before nutrients are depleted and growth stops.

As used herein, the term “edible food product” refers to a food product safe for human consumption. For example, this includes, but is not limited to a food product that is generally recognized as safe per a government or regulatory body (such as the United States Food and Drug Administration). In certain embodiments, the food product is considered safe to consume by a person of skill. Any edible food product suitable for a human consumption should also be suitable for consumption by another animal and such an embodiment is intended to be within the scope herein.

As used herein, the term “enzyme” or “enzymatically” refers to biological catalysts. Enzymes accelerate, or catalyze, chemical reactions. Enzymes increase the rate of reaction by lowering the activation energy.

As used herein, the term “expression” is the process by which information from a gene is used in the synthesis of a functional gene product.

As used herein, the term “fed-batch culture” refers to an operational technique where one or more nutrients, such as substrates, are fed to a bioreactor in continuous or periodic mode during cultivation and in which product(s) remain in the bioreactor until the end of a run. An alternative description is that of a culture in which a base medium supports initial cell culture and a feed medium is added to prevent nutrient depletion. In a fed-batch culture one can control concentration of fed-substrate in the culture liquid at desired levels to support continuous growth.

As used herein, a “gene product” is the biochemical material, either RNA or protein, resulting from expression of a gene.

As used herein, “growth medium” refers to a medium or culture medium that supports the growth of microorganisms or cells or small plants. A growth medium may be, without limitation, solid or liquid or semi-solid. Growth medium shall also be synonymous with “growth media.”

As used herein, “basal medium” refers to a non-supplemented medium which promotes the growth of many types of microorganisms and/or cells which do not require any special nutrient supplements.

As used herein, “in vitro” refers to a process performed or taking place in a test tube, culture dish, bioreactor, or elsewhere outside a living organism. In the body of this disclosure, a product may also be referred to as an in vitro product, in which case in vitro shall be an adjective and the meaning shall be that the product has been produced with a method or process that is outside a living organism.

As used herein, “suspension culture” refers to a type of culture in which single cells or small aggregates of cells multiply while suspended in agitated liquid medium. It also refers to a cell culture or a cell suspension culture.

As used herein, “fibroblasts” refers to mesenchymal-derived cells that are responsible for the extracellular matrix, epithelial differentiation, and regulation of inflammation and wound healing. In addition, fibroblasts are also responsible for the secretion of growth factors and work as scaffolds for other cell types. Fibroblasts are one cell type found in conventional meat.

As used herein, “cell paste” refers to a paste of cells harvested from a cell culture that contains water. The dry cell weight of cell paste can be 1%-5%, 5%-10%, 10%-15%, 15%-20%, 20%-25%, 25%-30%, 30%-35%, 35%-40%, 40%-45%, 45%-50%, or higher. A skilled worker can prepare cell paste with a desired water content. Typically, cell paste comprises about 5%-15% cells by dry cell weight. It is within the ambit of skilled practitioners to prepare cell paste that comprises a desired dry cell weight of cultivated cells, including cell paste that comprises any other desired percentage by dry cell weight. The skilled worker can remove moisture by centrifugation, lyophilization, heating or any other well-known drying techniques. According to the United States Department of Agriculture, the naturally occurring moisture content of animal meats including poultry, is about 75% water. In some embodiments, the cell paste provided herein comprises a significant amount of water. “Wet cell paste” as used herein comprises about 25%-90% water 25%-85% water, 25%-80% water, 25%-75% water, 25%-70% water, 25%-65% water, 25%-60% water, 25%-55% water, 25%-50% water, 30%-90% water, 30%-85% water, 30%-80% water, 30%-75% water, 30%-70% water, 30%-65% water, 30%-60% water, 30%-55% water, 30%-50% water, 35%-90% water, 35%-85% water, 35%-80% water, 35%-75% water, 35%-70% water, 35%-65% water, 35%-60% water, 35%-55% water, 35%-50% water, 40%-90% water, 40%-85% water, 40%-80% water, 40%-75% water, 40%-70% water, 40%-65% water, 40%-60% water, 40%-60% water, 40%-55% water, 40%-50% water, 45%-90% water, 45%-85% water, 45%-80% water, 45%-75% water, 45%-70% water, 45%-75% water, 45%-70% water, 45%-65% water, 45%-60% water, 45%-55% water, 45%-50% water, 50%-90% water, 50%-85% water, 50%-80% water, 50%-75% water, 50%-70% water, 50%-65% water, 50%-60% water, 50%-55% water. Cell paste is another term for cultured cell meat.

As used herein, “substantially pure” refers to cells that are at least 80% cells by dry weight. Substantially pure cells are between 80%-85% cells by dry weight, between 85%-90% cells by dry weight, between 90%-92% cells by dry weight, between 92%-94% cells by dry weight, between 94%-96% cells by dry weight, between 96%-98% cells by dry weight, between 98%-99% cells by dry weight.

As used herein, “seasoning” refers to one or more herbs and spices in both solid and liquid form.

As used herein, “primary avian fibroblast cells” refers to cells from a parental animal that maintain growth in a suitable growth medium, for instance under controlled environmental conditions. Cells in primary culture have the same karyotype (number and appearance of chromosomes in the nucleus of a eukaryotic cell) as those cells in the original tissue.

As used herein, “secondary avian fibroblast cells” refers to primary cells that have undergone a genetic transformation and become immortalized allowing for indefinite proliferation.

As used herein, “proliferation” refers to a process that results in an increase in the number of cells. It is characterized by a balance between cell division and cell loss through cell death or differentiation.

As used herein, “adventitious” refers to one or more contaminants such as, but not limited to: viruses, bacteria, mycoplasma, and fungi.

As used herein “peptide cross-linking enzyme” or “cross-linking enzyme is an enzyme that catalyzes the formation of covalent bonds between one or more polypeptides.

As used herein, “transglutaminase” or “TG” refers to an enzyme (R-glutamyl-peptide amine glutamyl transferase) that catalyzes the formation of a peptide (amide) bond between γ-carboxyamide groups and various primary amines, classified as EC 2.3.2.13. Transglutaminases catalyze the formation of covalent bonds between polypeptides, thereby cross-linked polypeptides. Cross-linking enzymes such as transglutaminase are used in the food industry to improve texture of some food products such as dairy, meat and cereal products. It can be isolated from a bacterial source, a fungus, a mold, a fish, a mammal, or a plant.

As used herein “protein concentrate” is a collection of one or more different polypeptides obtained from a plant source or animal source. The percent protein by dry weight of a protein concentrate is greater than 25% protein by dry weight.

As used herein “protein isolate” is a collection of one or more different polypeptides obtained from a plant source or an animal source. The percent protein by dry weight of a protein concentrate is greater than 50% protein by dry weight.

As used herein, and unless otherwise indicated, percentage (%) refers to total % by weight typically on a dry weight basis unless otherwise indicated.

The term “about” indicates and encompasses an indicated value and a range above and below that value. In certain embodiments, the term “about” indicates the designated value±10%, ±5%, or ±1%. In certain embodiments, the term “about” indicates the designated value±one standard deviation of that value.

In this disclosure, methods are presented for culturing avian derived cells in vitro. The methods herein provide methods to proliferate, recover, and monitor the purity of cell cultures. The cells can be used, for example, in one or more food products.

The disclosure herein sets forth embodiments for avian food products compositions comprising avian derived cells grown in vitro. In some embodiments, the compositions comprise plant protein, cell paste, fat, water, and a peptide cross-linking enzyme.

The disclosure herein sets forth embodiments for methods to prepare an avian food product made from avian derived cells grown in vitro. The avian food product is an edible food product.

### Cells

Provided herein are food products or processes comprising cells. In some embodiments, the cells are avian cells. In some embodiments, the avian cells are selected from, but not limited to: chicken, pheasant, goose, swan, pigeon, turkey, and duck. In some embodiments, the cells comprise primary avian fibroblast cells. In some embodiments, the cells comprise secondary avian fibroblast cells.

In some embodiments, the cells are UMNSAH/DF1 (C1F) cells. In certain embodiments, the cells are a commercially available chicken cell line deposited at American Type Culture Collection (ATCC, Manassas, Va., USA) on Oct. 11, 1996. In some embodiments, the cells used are derived from ATCC deposit number CRL12203.

In some embodiments, the avian cell lines have a spontaneously immortalized fibroblast phenotype. In some embodiments, the avian cell lines have high proliferation rates. In certain embodiments, the cells have both an immortalized fibroblast phenotype and high proliferation rates.

In some embodiments, the cells are not recombinant or engineered in any way (i.e., non-GMO). In some embodiments, the cells have not been exposed to any viruses and/or viral DNA. In certain embodiments, the cells are both not recombinant or have not been exposed to any viruses and/or viral DNA and/or RNA.

### Culture Media and Growth

In some embodiments, proliferation occurs in suspension or adherent conditions, with or without feeder-cells and/or in serum-containing or serum-free media conditions. In some embodiments, media for proliferation contains one or more of amino acids, peptides, proteins, carbohydrates, essential metals, minerals, vitamins, buffering agents, anti-microbial agents, growth factors, and/or additional components.

In some embodiments, proliferation is measured by any method known to one skilled in the art. In some embodiments, proliferation is measured through direct cell counts. In certain embodiments, proliferation is measured by a haemocytometer. In some embodiments, proliferation is measured by automated cell imaging. In certain embodiments, proliferation is measured by a Coulter counter.

In some embodiments, proliferation is measured by using viability stains. In certain embodiments, the stains used comprise trypan blue.

In some embodiments, proliferation is measured by the total DNA. In some embodiments, proliferation is measured by BrdU labelling. In some embodiments, proliferation is measured by metabolic measurements. In certain embodiments, proliferation is measured by using tetrazolium salts. In certain embodiments, proliferation is measured by ATP-coupled luminescence.

In some embodiments, the culture media is basal media. In some embodiments, the basal media is DMEM, DMEM/F12, MEM, HAMS's F10, HAM's F12, IMDM, McCoy's Media and RPMI.

In some embodiments, the basal media comprises amino acids. In some embodiments, the basal media comprises biotin. In some embodiments, the basal media comprises choline chloride. In some embodiments, the basal media comprises D-calcium pantothenate. In some embodiments, the basal media comprises folic acid. In some of embodiments, the basal media comprises niacinamide. In some embodiments, the basal media comprises pyridoxine hydrochloride. In some embodiments, the basal media comprises riboflavin. In some embodiments, thiamine hydrochloride is part of the basal media (DMEM/F12). In some embodiments, the basal media comprises vitamin B12 (also known as cyanocobalamin). In some embodiments, the basal media comprises i-inositol (myo-inositol). In some embodiments, the basal media comprises calcium chloride. In some embodiments, the basal media comprises cupric sulfate. In some embodiments, the basal media comprises ferric nitrate. In some embodiments, the basal media comprises magnesium chloride. In some embodiments, the basal media comprises magnesium sulfate. In some embodiments, the basal media comprises potassium chloride. In some embodiments, the basal media comprises sodium bicarbonate. In some embodiments, the basal media comprises sodium chloride. In some embodiments, the basal media comprises sodium phosphate dibasic. In some embodiments, the basal media comprises sodium phosphate monobasic. In some embodiments, the basal media comprises zinc sulfate. In some embodiments, the growth medium comprises sugars. In some embodiments, the sugars include but are not limited to D-glucose, galactose, fructose, mannose, or any combination thereof. In an embodiment, the sugars includes both D-glucose and mannose. In embodiments where glucose and mannose are both used in the growth medium to cultivate cells, the amount of glucose in the growth medium (cultivation media) is between 0.1-10 g/L, 0.1-9 g/L, 0.1-8 g/L, 0.1-7 g/L, 0.1-6 g/L, 0.1-5 g/L, 0.1-4 g/L, 0.1-3 g/L, 0.1-2 g/L, 0.1-1 g/L, 0.5-10 g/L, 0.5-9 g/L, 0.5-8 g/L, 0.5-7 g/L, 0.5-6 g/L, 0.5-5 g/L, 0.5-4 g/L, 0.5-3 g/L, 0.5-2 g/L, 0.5-1 g/L, 1-10 g/L, 1-9 g/L, 1-8 g/L, 1-9 g/L, 1-8 g/L, 1-7 g/L, 1-6 g/L, 1-5 g/L, 1-4 g/L, 1-3 g/L, 1-2 g/L, 2-10 g/L, 2-9 g/L, 2-8 g/L, 2-9 g/L, 2-8 g/L, 2-7 g/L, 2-6 g/L, 2-5 g/L, 2-4 g/L, 2-3 g/L, 3-10 g/L, 3-9 g/L, 3-8 g/L, 3-9 g/L, 3-8 g/L, 3-7 g/L, 3-6 g/L, 3-5 g/L, 3-4 g/L, 4-10 g/L, 4-9 g/L, 4-8 g/L, 4-9 g/L, 4-8 g/L, 4-7 g/L, 4-6 g/L, 4-5 g/L, 5-10 g/L, 5-9 g/L, 5-8 g/L, 5-9 g/L, 5-8 g/L, 5-7 g/L, or 5-6 g/L, and the amount of mannose in the growth media is between 0.1-10 g/L, 0.1-9 g/L, 0.1-8 g/L, 0.1-7 g/L, 0.1-6 g/L, 0.1-5 g/L, 0.1-4 g/L, 0.1-3 g/L, 0.1-2 g/L, 0.1-1 g/L, 0.5-10 g/L, 0.5-9 g/L, 0.5-8 g/L, 0.5-7 g/L, 0.5-6 g/L, 0.5-5 g/L, 0.5-4 g/L, 0.5-3 g/L, 0.5-2 g/L, 0.5-1 g/L, 1-10 g/L, 1-9 g/L, 1-8 g/L, 1-9 g/L, 1-8 g/L, 1-7 g/L, 1-6 g/L, 1-5 g/L, 1-4 g/L, 1-3 g/L, 1-2 g/L, 2-10 g/L, 2-9 g/L, 2-8 g/L, 2-9 g/L, 2-8 g/L, 2-7 g/L, 2-6 g/L, 2-5 g/L, 2-4 g/L, 2-3 g/L, 3-10 g/L, 3-9 g/L, 3-8 g/L, 3-9 g/L, 3-8 g/L, 3-7 g/L, 3-6 g/L, 3-5 g/L, 3-4 g/L, 4-10 g/L, 4-9 g/L, 4-8 g/L, 4-9 g/L, 4-8 g/L, 4-7 g/L, 4-6 g/L, 4-5 g/L, 5-10 g/L, 5-9 g/L, 5-8 g/L, 5-9 g/L, 5-8 g/L, 5-7 g/L, or 5-6 g/L. The skilled worker will understand that combinations of these amounts of glucose and mannose can be used, for example, between 2-5 grams of glucose and 1-4 grams of mannose.

In some embodiments, the basal media comprises linoleic acid. In some embodiments, the basal media comprises lipoic acid. In some embodiments, the basal media comprises putrescine-2HCl. In some embodiments, the basal media comprises 1,4 butanediamine. In some embodiments, the basal media comprises Pluronic F-68. In some embodiments, the basal media comprises fetal bovine serum. In certain embodiments, the basal media comprises each ingredient in this paragraph. In certain embodiments, the basal media is DMEM/F12.

In some embodiments, the growth medium comprises serum. In some embodiments, the serum is selected from bovine calf serum, chicken serum, and any combination thereof.

In some embodiments, the growth medium comprises at least 10% fetal bovine serum. In certain embodiments, the population of avian fibroblast cells are grown in a medium with at least 10% fetal bovine serum, followed by a reduction to less than 2% fetal bovine serum before recovering the cells.

In another embodiment, the culture media contains no serum including fetal bovine serum, fetal calf serum, or any animal derived serum.

In certain embodiments, the fetal bovine serum is reduced to less than or equal to 1.9% fetal bovine serum before recovering the cells. In certain embodiments, the fetal bovine serum is reduced to less than or equal to 1.7% fetal bovine serum before recovering the cells. In certain embodiments, the fetal bovine serum is reduced to less than or equal to 1.5% fetal bovine serum before recovering the cells. In certain embodiments, the fetal bovine serum is reduced to less than or equal to 1.3% fetal bovine serum before recovering the cells. In certain embodiments, the fetal bovine serum is reduced to less than or equal to 1.1% fetal bovine serum before recovering the cells. In certain embodiments, the fetal bovine serum is reduced to less than or equal to 0.9% fetal bovine serum before recovering the cells. In certain embodiments, the fetal bovine serum is reduced to less than or equal to 0.7% fetal bovine serum before recovering the cells. In certain embodiments, the fetal bovine serum is reduced to less than or equal to 0.5% fetal bovine serum before recovering the cells. In certain embodiments, the fetal bovine serum is reduced to less than or equal to 0.3% fetal bovine serum before recovering the cells. In certain embodiments, the fetal bovine serum is reduced to less than or equal to 0.1% fetal bovine serum before recovering the cells. In certain embodiments, the fetal bovine serum is reduced to less than or equal to 0.05% fetal bovine serum before recovering the cells. In certain embodiments, the fetal bovine serum is reduced to about 0% fetal bovine serum before recovering the cells.

In some embodiments, the basal media is DMEM/F12 and is in a ratio of 3:1; 2:1; or 1:1. In certain embodiments, the basal media is DMEM/F12 and in a ratio of about 3:1. In certain embodiments, the basal media is DMEM/F12 and in a ratio of about 2:1. In certain embodiments, the basal media is DMEM/F12 and in a ratio of about 1:1.

In some embodiments, the growth media is modified in order to optimize the expression of at least one gene from a cell signaling pathway selected from the group consisting of proteasome, steroid biosynthesis, amino acid degradation, amino acid biosynthesis, drug metabolism, focal adhesion, cell cycle, MAPK signaling, glutathione metabolism, TGF-beta, phagosome, terpenoid biosynthesis, DNA replication, glycolysis, gluconeogenesis, protein export, butanoate metabolism, and synthesis and degradation of ketone bodies.

In some embodiments, the steps of producing avian fibroblast are monitored for gene expression of one or more cell signaling pathways. In certain embodiments, the growth media is adjusted at each stage of cell production in accordance with data obtained from the monitoring of gene expression.

In some embodiments, the avian fibroblast cells are induced to accumulate lipids by adding or removing one or more compounds to or from the growth media in quantities sufficient to induce the accumulation of one or more lipids.

In some embodiments, one or more of the maintenance, proliferation, differentiation, lipid accumulation, lipid content, proneness to purification and/or harvest efficiency, growth rates, cell densities, cell weight, resistance to contamination, avian fibroblast-specific gene expression and/or protein secretion, shear sensitivity, flavor, texture, color, odor, aroma, gustatory quality, nutritional quality, minimized growth-inhibitory byproduct secretion, and/or minimized media requirements, of avian fibroblast cells, in any culture conditions, are improved by one or more of growth factors, proteins, peptides, fatty acids, elements, small molecules, plant hydrosylates, directed evolution, genetic engineering, media composition, bioreactor design, and/or scaffold design. In certain embodiments, the fatty acids comprise stearidonic acid (SDA). In certain embodiments, the fatty acids comprise linoleic acid. In certain embodiments, the growth factor comprises insulin or insulin like growth factor. In certain embodiments, the growth factor comprises fibroblast growth factor or the like. In certain embodiments, the growth factor comprises epidermal growth factor or the like. In certain embodiments, the protein comprises transferrin. In certain embodiments, the element comprises selenium. In certain embodiments, a small molecule comprises ethanolamine. The amount of ethanolamine used in the cultivations is between 0.05-10 mg/L, 0.05-10 mg/L, 0.1-10 mg/L, 0.1-9.5 mg/L, 0.1-9 mg/L, 0.1-8.5 mg/L, 0.1-8.0 mg/L, 0.1-7.5 mg/L, 0.1-7.0 mg/L, 0.1-6.5 mg/L, 0.1-6.0 mg/L, 0.1-5.5 mg/L, 0.1-5.0 mg/L, 0.1-4.5 mg/L, 0.1-4.0 mg/L, 0.1-3.5 mg/L, 0.1-3.0 mg/L, 0.1-2.5 mg/L, 0.1-2.0 mg/L, 0.1-1.5 mg/L, and 0.1-1.0 mg/L.

In certain embodiments, the media can be supplemented with plant hydrolysates. In certain embodiments, the hydrolysates comprise yeast extract, wheat peptone, rice peptone, phytone peptone, yeastolate, pea peptone, soy peptone, pea peptone, potato peptone, mung bean protein hydrolysate, or sheftone. The amount of hydrolysate used in the cultivations is between 0.1 g/L to 5 g/L, between 0.1 g/L to 4.5 g/L, between 0.1 g/L to 4 g/L, between 0.1 g/L to 3.5 g/L, between 0.1 g/L to 3 g/L, between 0.1 g/L to 2.5 g/L, between 0.1 g/L to 2 g/L, between 0.1 g/L to 1.5 g/L, between 0.1 g/L to 1 g/L, or between 0.1 g/L to 0.5 g/L.

In some embodiments, a small molecule comprises lactate dehydrogenase inhibitors. As described in the Examples below, lactate dehydrogenase inhibitors inhibit the formation of lactate. The production of lactate by avian cells inhibit the growth of the cells. Exemplary lactate dehydrogenase inhibitors are selected from the group consisting of oxamate, galloflavin, gossypol, quinoline 3-sulfonamides, N-hydroxyindole-based inhibitors, and FX11. In some embodiments, the amount of lactate dehydrogenase inhibitor in the fermentation medium is between 1-500 mM, 1-400 mM, 1-300 mM, 1-250 mM, between 1-200 mM, 1-175 mM, 1-150 mM, 1-100 mM, 1-50 mM, 1-25 mM, 25-500 mM, 25-400 mM, 25-300 mM, 25-250 mM, 25-200 mM, 25-175 mM, 25-125M, 25-100 mM, 25-75 mM, 25-50 mM, 50-500 mM, 50-400 mM, 50-300 mM, 50-250 mM, 50-200 mM, 50-175 mM, 50-150 mM, 50-125 mM, 50-100 mM, 50-75 mM, 75-500 mM, 75-400 mM, 75-300 mM, 75-250 mM, 75-200 mM, 75-175 mM, 75-150 mM, 75-125 mM, 75-100 mM, 100-500 mM, 100-400 mM, 100-300 mM, 100-250 mM, 100-200 mM, 100-150 mM, 100-125 mM, and 100-500 mM.

In some embodiments, the avian fibroblast cells are grown in a suspension culture system. In some embodiments, the avian fibroblast cells are grown in a batch, fed-batch, semi continuous (fill and draw) or perfusion culture system or some combination thereof. When grown in suspension culture, the suspension culture can be performed in a vessel (fermentation tank, bioreactor)) of a desired size. The vessel is a size that is suitable for growth of avian cells without unacceptable rupture of the cells. In some embodiments, the suspension culture system can be performed in vessel that is at least 25 liters (L), 50 L, 100 L, 200 L, 250 L, 350 L, 500 liters (L), 1000 L, 2,500 L, 5,000 L, 10,000 L, 25,000 L, 50,000 L, 100,000 L, 200,000 L, 250,000 L, or 500,000 L. For smaller suspension cultures, the cultivation of the cells can be performed in a flask that is least 125 mL, 250 mL, 500 mL, 1 L, 1.5 L, 2 L, 2.5 L, 3 L, 5 L, 10 L, or larger.

In some embodiments, the cell density of the suspension culture is between 0.25×10⁶ cells·ml, 0.5×10⁶ cells/ml and 1.0×10⁶ cells/ml, between 1.0×10⁶ cells/ml and 2.0×10⁶ cells/ml, between 2.0×10⁶ cells/ml and 3.0×10⁶ cells/ml, between 3.0×10⁶ cells/ml and 4.0×10⁶ cells/ml, between 4.0×10⁶ cells/ml and 5.0×10⁶ cells/ml, between 5.0×10⁶ cells/ml and 6.0×10⁶ cells/ml, between 6.0×10⁶ cells/ml and 7.0×10⁶ cells/ml, between 7.0×10⁶ cells/ml and 8.0×10⁶ cells/ml, between 8.0×10⁶ cells/ml and 9.0×10⁶ cells/ml, between 9.0×10⁶ cells/ml and 10×10⁶ cells/ml, between 10×10⁶ cells/ml and 15.0x×10⁶ cells/ml, between 15x×10⁶ cells/ml and 20x×10⁶ cells/ml, between 20x×10⁶ cells/ml and 25×10⁶ cells/ml, between 25×10⁶ cells/ml and 30×10⁶ cells/ml, between 30×10⁶ cells/ml and 35×10⁶ cells/ml, between 35×10⁶ cells/ml and 40×10⁶ cells/ml, between 40×10⁶ cells/ml and 45×10⁶ cells/ml, between 45×10⁶ cells/ml and 50×10⁶ cells/ml, between 50×10⁶ cells/ml and 55×10⁶ cells/ml, between 55×10⁶ cells/ml and 60×10⁶ cells/ml, between 60×10⁶ cells/ml and 65×10⁶ cells/ml, between 70×10⁶ cells/ml and 75×10⁶ cells/ml, between 75×10⁶ cells/ml and 80×10⁶ cells/ml, between 85×10⁶ cells/ml and 90×10⁶ cells/ml, between 90×10⁶ cells/ml and 95×10⁶ cells/ml, between 95×10⁶ cells/ml and 100×10⁶ cells/ml, between 100×10⁶ cells/ml and 125×10⁶ cells/ml, or between 125×10⁶ cells/ml and 150×10⁶ cells/ml.

In some embodiments, the avian fibroblast cells are grown while embedded in scaffolds or attached to scaffolding materials. In some embodiments, the avian fibroblast cells are differentiated or proliferated in a bioreactor and/or on a scaffold. In some embodiments, the scaffold comprises at least one or more of a microcarrier, an organoid and/or vascularized culture, self-assembling co-culture, a monolayer, hydrogel scaffold, decellularized avian fibroblasts and/or an edible matrix. In some embodiments, the scaffold comprises at least one of plastic and/or glass or other material. In some embodiments, the scaffold comprises natural-based (biological) polymers chitin, alginate, chondroitin sulfate, carrageenan, gellan gum, hyaluronic acid, cellulose, collagen, gelatin, and/or elastin. In some embodiments, the scaffold comprises a protein or a polypeptide, or a modified protein or modified polypeptide. The unmodified protein or polypeptide or modified protein or polypeptide comprises proteins or polypeptides isolated from plants or other organisms. Exemplary plant protein isolates or plant protein concentrates comprise pulse protein, vetch protein, grain protein, nut protein, macroalgal protein, microalgal protein, and other plant proteins. Pulse protein can be obtained from dry beans, lentils, mung beans, faba beans, dry peas, chickpeas, cowpeas, bambara beans, pigeon peas, lupins, vetches, adzuki, common beans, fenugreek, long beans, lima beans, runner beans, or tepary beans, soybeans, or mucuna beans. Vetch protein can be obtained from the genus Vicia. Grain protein can be obtained from wheat, rice, teff, oat, corn, barley, sorghum, rye, millet, triticale, amaranth, buckwheat, quinoa and other grains. Nut protein can be obtained from almond, cashew, pecan, peanut, walnut, macadamia, hazelnut, pistachio, brazil, chestnut, kola nut, sunflower seeds, pumpkin seeds, flax seeds, cacao, pine nut, ginkgo, and other nuts. Proteins obtained from animal source can also be used as scaffolds, including milk proteins, whey, casein, egg protein, and other animal proteins. In some embodiments, the self-assembling co-cultures comprise spheroids and/or aggregates. In some embodiments, the monolayer is with or without an extracellular matrix. In some embodiments, the hydrogel scaffolds comprise at least one of hyaluronic acid, alginate and/or polyethylene glycol. In some embodiments, the edible matrix comprises decellularized plant tissue.

In some embodiments, either primary or secondary avian fibroblast cells are modified or grown as in any of the preceding paragraphs.

### Recovery of Cells

The cells can be recovered by any technique apparent to those of skill. In some embodiments the avian fibroblast cells are separated from the growth media or are removed from a bioreactor or a scaffold. In certain embodiments, the avian fibroblast cells are separated by centrifugation, a mechanical/filter press, filtration, flocculation or coagulation or gravity settling or drying or some combination thereof. In certain embodiments, the filtration method comprises tangential flow filtration, vacuum filtration, rotary vacuum filtration and similar methods. In certain embodiments the drying can be accomplished by flash drying, bed drying, tray drying and/or fluidized bed drying and similar methods. In certain embodiments, the avian fibroblasts are separated enzymatically. In certain embodiments, the avian fibroblasts are separated mechanically.

### Cell Safety

In some embodiments, the population of avian fibroblast is substantially pure.

In some embodiments, tests are administered at one or more steps of cell culturing to determine whether the avian fibroblast cells are substantially pure.

In some embodiments, the avian fibroblast cells are tested for the presence or absence of bacteria. In certain embodiments, the types of bacteria tested include, but are not limited to: Salmonella enteritidis, Staphylococcus aureus, Campylobacter jejunim, Listeria monocytogenes, Fecal streptococcus, Mycoplasma genus, Mycoplasma pulmonis, Coliforms, and Escherichia coli.

In some embodiments, components of the cell media, such as Fetal Bovine Serum, are tested for the presence or absence of viruses. In certain embodiments, the viruses include, but are not limited to: Bluetongue, Bovine Adenovirus, Bovine Parvovirus, Bovine Respiratory Syncytial Virus, Bovine Viral Diarrhea Virus, Rabies, Reovirus, Adeno-associated virus, BK virus, Epstein-Barr virus, Hepatitis A virus, Hepatitis B virus, Hepatitis C virus, Herpes Simplex 1, Herpes Simplex 2, Herpes virus type 6, Herpes virus type 7, Herpes virus type 8, HIV1, HIV-2, HPV-16, HPV 18, Human cytomegalovirus, Human Foamy virus, Human T-lymphotropic virus, John Cunningham virus, and Parvovirus B19.

In some embodiments, the tests are conducted for the presence or absence of yeast and/or molds.

In some embodiments, the tests are for metal concentrations by mass spectrometry, for example inductively coupled plasma mass spectrometry (ICP-MS). In certain embodiments, metals tested include, but are not limited to: arsenic, lead, mercury, cadmium, and chromium.

In some embodiments, the tests are for hormones produced in the culture. In certain embodiments, the hormones include, but are not limited: to 17β-estradiol, testosterone, progesterone, zeranol, melengesterol acetate, trenbolone acetate, megestrol acetate, melengesterol acetate, chlormadinone acetate, dienestrol, diethylstilbestrol, hexestrol, taleranol, zearalanone, and zeranol.

In some embodiments, the tests are in keeping with the current good manufacturing process as detailed by the United States Food and Drug Administration.

### Phenotyping, Process Monitoring and Data Analysis

In some embodiments, the cells are monitored by any technique known to a person of skill in the art. In some embodiments, differentiation is measured and/or confirmed using transcriptional markers of differentiation after total RNA extraction using RT-qPCR and then comparing levels of transcribed genes of interest to reference, e.g. housekeeping, genes.

### Food Composition

In certain embodiments provided herein are food compositions or food products comprising avian fibroblast cells. In some embodiments, the avian fibroblast cells are combined with other substances or ingredients to make a composition that is an avian food product composition. In certain embodiments, the avian fibroblast cells are used alone to make a composition that is an avian food product composition. In certain embodiments, the avian food product composition is a product that resembles: avian nuggets, avian tenders, avian breasts, avian oysters, avian feet, avian wings, avian sausage, avian feed stock, or avian skin. In certain embodiments, the avian product resembles a chicken product.

In some embodiments, the recovered avian fibroblast cells are prepared into a composition with other ingredients. In certain embodiments, the composition comprises cell paste, mung bean, fat, and water.

In certain embodiments, the food composition or food product has a wet cell paste content of at least 100%, 90%, 80%, 75%, 70%, 65%, 60%, 50%, 40%, 30%, 35%, 25%, 15%, 10%, 5% or 1% by weight. In certain embodiments, the food composition or food product has a wet cell paste content by weight of between 10%-20%, 20%-30%, 30%-40%, 40%-50%, 60%-70%, 80%-90%, or 90%-100%. In certain embodiments, the composition comprises a pulse protein content by weight of at least 75%, 70%, 60%, 50%, 40%, 30%, 25%, 20%, or 15% by weight. In certain embodiments, the food composition or food product has a pulse protein content by weight of between 10%-20%, 20%-30%, 30%-40%, 40%-50%, 60%-70%, 80%-90%, or 90%-95%. In certain embodiments, the food composition or food product comprises a fat content of at least 50%, 40%, 30%, 25%, 20%, 15%, 10%, 5%, or 1% by weight. In certain embodiments, the food composition or food product has a fat content by weight of between 10%-20%, 20%-30%, 30%-40%, 40%-50%, 60%-70%, 80%-90%, or 90%-95%. In certain embodiments, the food composition or food product comprises a water content of at least 50%, 40%, 30%, 25%, 20%, 15%, 10% or 5% by weight. In certain embodiments, the food composition or food product has a water content by weight of between 10%-20%, 20%-30%, 30%-40%, 40%-50%, 60%-70%, 80%-90%, or 90-95%. In certain embodiments, the food composition or food product comprises a wet cell paste content of between 2%-5%, 5%-10%, 10%-15%, 15%-20%, 20%-25%, 25%-30%, 30%-35%, 35%-40%, 40%-45%, 45%-50%, 50%-55%, 55%-60%, 65%-70%, 70%-75%, 75%-80%, 80%-85%, 85%-90%, or 90%-95%.

In some embodiments, the composition comprises a peptide cross-linking enzyme, for example, transglutaminase content between 0.0001-0.0125%.

In certain embodiments, the food composition or food product comprises a dry cell weight content of at least of 1% by weight. In certain embodiments, the food composition or food product comprises a dry cell weight content of at least of 5% by weight. In certain embodiments, the food composition or food product comprises a dry cell weight content of at least of 10% by weight. In certain embodiments, the food composition or food product comprises a dry cell weight content of at least of 15% by weight. In certain embodiments, the food composition or food product comprises a dry cell weight content of at least of 20% by weight. In certain embodiments, the food composition or food product comprises a dry cell weight content of at least of 25% by weight. In certain embodiments, the composition or food product comprises a dry cell weight of at least of 30% by weight. In certain embodiments, the composition or food product comprises a dry cell weight of at least of 35% by weight. In certain embodiments, the composition or food product comprises a dry cell weight of at least of 40% by weight. In certain embodiments, the composition or food product comprises a dry cell weight of at least of 45% by weight. In certain embodiments, the composition or food product comprises a dry cell weight of at least of 50% by weight. In certain embodiments, the composition or food product comprises a dry cell weight of at least of 55% by weight. In certain embodiments, the composition or food product comprises a dry cell weight of at least of 60% by weight. In certain embodiments, the composition or food product comprises a dry cell weight of at least of 65% by weight. In certain embodiments, the composition or food product comprises a dry cell weight of at least of 70% by weight. In certain embodiments, the composition or food product comprises a dry cell weight of at least of 75% by weight. In certain embodiments, the composition or food product comprises a dry cell weight of at least of 80% by weight. In certain embodiments, the composition or food product comprises a dry cell weight of at least of 85% by weight. In certain embodiments, the composition or food product comprises a dry cell weight of at least of 90% by weight. In certain embodiments, the composition or food product comprises a dry cell weight of at least of 95% by weight. In certain embodiments, the composition or food product comprises a dry cell weight of at least of 97% by weight. In certain embodiments, the composition or food product comprises a dry cell weight of at least of 98% by weight. In certain embodiments, the composition or food product comprises a dry cell weight of at least of 99% by weight. In certain embodiments, the composition or food product comprises a dry cell weight of at least of 100% by weight. In certain embodiments, the food composition or food product comprises a dry cell weight content of between 2%-5%, 5%-10%, 10%-15%, 15%-20%, 20%-25%, 25%-30%, 30%-35%, 35%-40%, 40%-45%, 45%-50%, 50%-55%, 55%-60%, 65%-70%, 70%-75%, 75%-80%, 80%-85%, 85%-90%, or 90%-95%,

In certain embodiments, the food composition or food product comprises a pulse protein content of at least 2%, 5%, 10%, 15%, 20%, 25%, 30%, 35%, 40%, 45%, 50%, 55%, 60%, 65%, 70%, 75%, 80%, 85%, 90% or 95% by weight. In certain embodiments, the food composition or food product comprises a pulse protein content of between 2%-5%, 5%-10%, 10%-15%, 15%-20%, 20%-25%, 25%-30%, 30%-35%, 35%-40%, 40%-45%, 45%-50%, 50%-55%, 55%-60%, 65%-70%, 70%-75%, 75%-80%, 80%-85%, 85%-90%, or 90%-95%, In some embodiments, the pulse protein is a mung bean protein.

In certain embodiments, the food composition or food product comprises, a fat content of at least 1% by weight, a fat content of at least 2% by weight, a fat content of at least 5% by weight, a fat content of at least 7.5% by weight, or a fat content of at least 10% by weight. In certain embodiments, the food composition or food product comprises a fat content of at least 15% by weight. In certain embodiments, the food composition or food product comprises a fat content of at least 20% by weight. In certain embodiments, the food composition or food product comprises a fat content of at least 25% by weight. In certain embodiments, the food composition or food product comprises a fat content of at least 27% by weight. In certain embodiments, the food composition or food product comprises a fat content of at least 30% by weight. In certain embodiments, the food composition or food product comprises a fat content of at least 35% by weight. In certain embodiments, the food composition or food product comprises a fat content of at least 40% by weight. In certain embodiments, the food composition or food product comprises a fat content of at least 45% by weight. In certain embodiments, the food composition or food product comprises a fat content of at least 50% by weight. In certain embodiments, the food composition or food product comprises a fat content of at least 55% by weight. In certain embodiments, the food composition or food product comprises a fat content of at least 60% by weight. In certain embodiments, the food composition or food product comprises a fat content of at least 65% by weight. In certain embodiments, the food composition or food product comprises a fat content of at least 70% by weight. In certain embodiments, the food composition or food product comprises a fat content of at least 75% by weight. In certain embodiments, the food composition or food product comprises a fat content of at least 80% by weight. In certain embodiments, the food composition or food product comprises a fat content of at least 85% by weight. In certain embodiments, the food composition or food product comprises a fat content of at least 90% by weight. In some embodiments, that food composition or food product comprises a fat content of between 1%-5%, between 5%-10%, between 10%-15%, between 15%-20%, between 20%-25%, between 25%-30%, between 30%-35%, between 35%-40%, between 45%-50%, between 50%-55%, between 55%-60%, between 60%-65%, between 65%-70%, between 70%-75%, between 75%-80%, between 80%-85%, between 85%-90%, or between 90%-95%.

In certain embodiments, the food composition or food product comprises a water content of at least 5% by weight. In certain embodiments, the food composition or food product comprises a water content of at least 10% by weight. In certain embodiments, the food composition or food product comprises a water to an amount of 15% by weight. In certain embodiments, the food composition or food product comprises a water content of at least 20% by weight. In certain embodiments, the food composition or food product comprises a water content of at least 25% by weight. In certain embodiments, the food composition or food product comprises a water content of at least 30% by weight. In certain embodiments, the food composition or food product comprises a water content of at least 35% by weight. In certain embodiments, the food composition or food product comprises a water content of at least 40% by weight. In certain embodiments, the food composition or food product comprises a water content of at least 45% by weight. In certain embodiments, the food composition or food product comprises a water content to an amount of 50% by weight. In certain embodiments, the food composition or food product comprises a water content to an amount of 55% by weight. In certain embodiments, the food composition or food product comprises a water content to an amount of 60% by weight. In certain embodiments, the food composition or food product comprises a water content to an amount of 65% by weight. In certain embodiments, the food composition or food product comprises a water content to an amount of 70% by weight. In certain embodiments, the food composition or food product comprises a water content to an amount of 75% by weight. In certain embodiments, the food composition or food product comprises a water content to an amount of 80% by weight. In certain embodiments, the food composition or food product comprises a water content to an amount of 85% by weight. In certain embodiments, the food composition or food product comprises a water content to an amount of 90% by weight. In certain embodiments, the food composition or food product comprises a water content to an amount of 95% by weight.

In one embodiment, the food composition or food product comprises a wet cell paste content between 25-75% by weight, a mung bean protein content between 15-45% by weight, a fat content between 10-30% by weight, and a water content between 20-50% by weight.

In certain embodiments, the food composition or food product comprises peptide cross-linking enzyme. Exemplary peptide cross-linking enzymes are selected from the group consisting of transglutaminase, sortase, subtilisin, tyrosinase, laccase, peroxidase, and lysyl oxidase. In certain embodiments, the composition comprises a cross-linking enzyme of between 0.0001%-0.025%, 0.0001%-0.020%, 0.0001%-0.0175%, 0.0001%-0.0150%, 0.0001%-0.0125%, 0.0001%-0.01%, 0.0001%-0.0075%, 0.0001%-0.005%, 0.0001%-0.0025%, 0.0001%-0.002%, 0.0001%-0.0015%, 0.0001%-0.001%, 0.0001%-0.00015% by weight. In certain embodiments, the food composition or food product comprises a transglutaminase content between 0.0001%-0.025%, 0.0001%-0.020%, 0.0001%-0.0175%, 0.0001%-0.0150%, 0.0001%-0.0125%, 0.0001%-0.01%, 0.0001%-0.0075%, 0.0001%-0.005%, 0.0001%-0.0025%, 0.0001%-0.002%, 0.0001%-0.0015%, 0.0001%-0.001%, 0.0001%-0.00015% by weight. Without being bound by theory, the peptide cross-linking enzyme is believed to cross-link the pulse or vetch proteins and the peptide cross-linking enzyme is believed to cross-link the pulse or vetch proteins to the avian cells.

In one embodiment, the food composition or food product comprises 0.0001% to 0.0125% transglutaminase, and exhibits reduced or significantly reduced lipoxygenase activity or other enzymes which oxidize lipids, as expressed on a volumetric basis relative to cell paste without the transglutaminase. More preferably, the food composition or food product is essentially free of lipoxygenase or enzymes that can oxidize lipids. In some embodiments, a 5%, 10%, 15%, 20%, 25%, 30%, 35%, 40%, 45%, 50%, 55%, 60%, 65%, 70%, 75%, or 80% reduction in oxidative enzymatic activity relative to a composition is observed. Lipoxygenases catalyze the oxidation of lipids that contribute to the formation of compounds that impart undesirable flavors to compositions.

In some embodiments, mung bean protein is replaced by plant-based protein comprising protein from garbanzo, fava beans, yellow pea, sweet brown rice, rye, golden lentil, chana dal, soybean, adzuki, sorghum, sprouted green lentil, du pung style lentil, and/or white lima bean.

In some embodiments, the addition of additional edible ingredients can be used to prepare the food composition of food product. Edible food ingredients comprise texture modifying ingredients such as starches, modified starches, gums and other hydrocolloids. Other food ingredients comprise pH regulators, anti-caking agents, colors, emulsifiers, flavors, flavor enhancers, foaming agents, anti-foaming agents, humectants, sweeteners, and other edible ingredients.

In certain embodiments, the methods and food composition or food product comprise an effective amount of an added preservative in combination with the food combination.

Preservatives prevent food spoilage from bacteria, molds, fungi, or yeast (antimicrobials); slow or prevent changes in color, flavor, or texture and delay rancidity (antioxidants); maintain freshness. In certain embodiments, the preservative is one or more of the following: ascorbic acid, citric acid, sodium benzoate, calcium propionate, sodium erythorbate, sodium nitrite, calcium sorbate, potassium sorbate, BHA, BHT, EDTA, tocopherols (Vitamin E) and antioxidants, which prevent fats and oils and the foods containing them from becoming rancid or developing an off-flavor.

### Food Process

In some embodiments, provided herein are processes for making an avian food product that comprises combining pulse protein, cell paste and a phosphate into water and heating up the mixture in three steps. In certain embodiments, the processes comprise adding phosphate to water thereby conditioning the water to prepare conditioned water. In certain embodiments, pulse protein is added to the conditioned water in order to hydrate the pulse protein to prepare hydrated plant protein. In some embodiments, cell paste is added to the hydrated plant protein (conditioned water to which a plant protein has been added) to produce a cell protein mixture. In some embodiments, the plant protein is a pulse protein. In some embodiments, the pulse protein is a mung bean protein

In some embodiments, the phosphate is selected from the group consisting of disodium phosphate (DSP), sodium hexametaphosphate (SHMP), tetrasodium pyrophosphate (TSPP). In one particular embodiment, the phosphate added to the water is DSP. In some embodiments, the amount of DSP added to the water is at least or about 0.01%, 0.02%, 0.03%, 0.04%, 0.05%, 0.06%, 0.07%, 0.08%, 0.09%, 0.1%, 0.11%, 0.12%, 0.13%, 0.14%, 0.15%, or greater than 0.15%.

In some embodiments, the process comprises undergo three heating steps. In some embodiments, the first heating step comprises heating the cell and protein mixture to a temperature between 40-65° C., wherein seasoning is added. In some embodiments, the second step comprises maintaining the cell and protein mixture at temperature between 40-65° C. for at least 10 minutes, wherein a peptide cross-linking enzyme such as transglutaminase is added. In some embodiments, the third heating step comprises raising the temperature of the cell and protein mixture to a temperature between 60-85° C., where oil is added to the water. In some embodiments, the process comprises a fourth step of lowering the temperature to a temperature between 5-15° C. to prepare a pre-cooking product.

In some embodiments, the seasonings are added to the first step, second step, third step or the fourth step. In some embodiments the seasonings include but are not limited to salt, sugar, paprika, onion powder, garlic powder, black pepper, white pepper, and natural chicken flavor (Vegan).

In some embodiments, the oil (fat) added is to the first step, second step, third step or the fourth step to prepare the pre-cooking product. The oil is selected from the group comprising vegetable oil, peanut oil, canola oil, coconut oil, olive oil, corn oil, soybean oil, sunflower oil, margarine, vegetable shortening, animal oil, butter, tallow, lard, margarine, or an edible oil.

In some embodiments, the pre-cooking product can be consumed without additional preparation or cooking, or the pre-cooking product can be cooked further, using well-known cooking techniques.

In some embodiments, the processes comprise preparing the avian food product by placement into cooking molds. In some embodiments, the processes comprise applying a vacuum to the cooking molds effectively changing the density and texture of the avian food product.

In some embodiments, the avian food product is breaded.

In some embodiments, the avian food product is steamed, boiled, sautéed, fried, baked, grilled, broiled, microwaved, dehydrated, cooked by sous vide, pressure cooked, or frozen or any combination thereof.

### Plant Protein Isolation

This application references and incorporates the methods for processing plant protein to produce plant protein concentrate and/or plant protein concentrate from US Publication No.: WO2013/067453, US 2017/0238590 A1, WO2017/143298, WO2017/143301, and U.S. 62/981,890 in their entirety.

Provided herein are methods for producing a plant protein isolate or plant protein concentrate having high functionality for a broad range of food applications. In some embodiments, the methods for producing the isolate comprise one or more steps selected from:

(a) extracting one or more or plant protein proteins from a plant protein source in an aqueous solution. In some embodiments, the extraction is performed at a pH between about 5.0-10.0.

(b) purifying protein from the extract using at least one of two methods:
(i) precipitating protein from the extract at a pH near the isoelectric point of a globulin-rich fraction, for example a pH between about 5.0-6.0; and/or
(ii) fractionating and concentrating protein from the extract using filtration methods such as microfiltration, ultrafiltration or chromatography.

(c) recovering purified protein isolate.

In particular embodiments, the plant protein isolate is produced using a series of mechanical processes, with the only chemicals used being pH adjusting agents, such as sodium hydroxide and citric acid, and optionally ethylenediaminetetraacetic acid (EDTA) to prevent lipid oxidation activities affecting the flavor of the isolate.

Although the plant protein isolates or plant protein concentrates provided herein may be prepared from any suitable source of plant protein, where the starting material is whole plant material such as whole mung bean, whole adzuki bean, pea or other plant material, a first step of the methods provided herein typically comprises dehulling the raw source material. In some such embodiments, raw beans are de-hulled in one or more steps of pitting, soaking, and drying to remove the seed coat (husk) and pericarp (bran). The de-hulled mung beans are then milled to produce flour with a well-defined particle distribution size. In some embodiments, the mean particle distribution size is less than 1000, 900, 800, 700, 600, 500, 400, 300, 200 or 100 μm. In a particular embodiment, the particle distribution size is less than 300 μm to increase the rate and yield of protein during the extraction step. The types of mills employed include but are not limited to one or a combination of a hammer, pin, knife, burr, and air classifying mills.

When feasible, air classification of the resultant flour may expedite the protein extraction process and enhance efficiency of the totality of the process. The method employed is to ensure the beans are milled to a particle size that is typically less than 45 μm, utilizing a fine-grinding mill, such as an air classifying mill. The resultant flour is then passed through an air classifier, which separates the flour into both a coarse and fine fraction. The act of passing the flour through the air classifier is intended to concentrate the majority of the available protein in the flour into a smaller portion of the total mass of the flour. Typical fine fraction (high-protein) yields are 5-50%. The fine fraction tends to be of a particle size of less than 20 μm; however, this may be influenced by growing season and region of the original bean. The high-protein fraction typically contains 150-220% of the protein in the original sample. The resultant starch-rich byproduct stream also becomes value added, and of viable, saleable interest as well.

In preferred embodiments, the methods to purify plant protein isolate or plant protein concentrate comprise an extraction step. In some embodiments of the extraction step, an intermediate starting material, for example, bean flour, is mixed with aqueous solution to form a slurry. In some embodiments, the aqueous solution is water, for example soft water. The aqueous extraction includes creating an aqueous solution comprising one part of the source of the plant protein (e.g., flour) to about, for example, 2 to 15 parts aqueous extraction solution. In other embodiments, 5 to 10 volumes of aqueous extraction solution is used per one part of the source of the plant protein. Additional useful ratios of aqueous extraction solution to flour include 1:1, 2:1, 4:1, 6:1, 7:1, 8:1, 9:1, 10:1, 11:1, 12:1, 13:1, 14:1, 15:1 or alternatively 1:2, 1:3, 1:4, 1:5, 1:6, 1:7, 1:8, 1:9, 1:10, 1:11, 1:12, 1:13, 1:14, 1:15.

Preferably, the aqueous extraction is performed at a desired temperature, for example, about 2-50° C. in a chilled mix tank to form the slurry. In some embodiments, the mixing is performed under moderate to high shear. In some embodiments, a food-grade de-foaming agent (e.g., KFO 402 Polyglycol) is added to the slurry to reduce foaming during the mixing process. In other embodiments, a de-foaming agent is not utilized during extraction.

In some embodiments, sequential extraction with multiple stages is performed to improve the extraction.

In some embodiments, the sequential extraction is performed either in batch mode or continuous mode

In some embodiments the sequential extraction is performed in current or counter current mode.

The pH of the slurry is adjusted with a food-grade 50% sodium hydroxide solution to reach the desired extraction pH for solubilization of the target protein into the aqueous solution. In some embodiments, the extraction is performed at a pH between about 5-10.0. In other embodiments, the extraction is performed at neutral or near neutral pH. In some embodiments, the extraction is performed at a pH of about pH 5.0-pH 9, pH 6.0-pH 8.5 or more preferably pH 6.5-pH 8. In a particular embodiment, the extraction is performed at a pH of about 6.5, 6.6, 6.7, 6.8, 6.9, 7.0, 7.1, 7.2, 7.3, 7.4, 7.5, 7.6, 7.7, 7.8, 7.9, 8.0, 8.1, 8.2, 8.3, 8.4, 8.5, 8.6, 8.7, 8.8, 8.9, 9.0, 9.1, 9.2, 9.3, 9.4, 9.5, 9.6, 9.7, 9.8, 9.9, or 10.0. In a particular embodiment, the extraction is performed at a pH of about 7.0.

Following extraction, the solubilized protein extract is separated from the slurry, for example, in a solid/liquid separation unit, consisting of a decanter and a disc-stack centrifuge. The extract is centrifuged at a low temperature, preferably between 3-10° C. The extract is collected, and the pellet is resuspended, preferably in 3:1 water-to-flour. The pH is adjusted again and centrifuged. Both extracts are combined and filtered through using a Nylon mesh.

Optionally, the protein extract is subjected to a carbon adsorption step to remove non-protein, off-flavor components, and additional fibrous solids from the protein extraction. This carbon adsorption step leads to a clarified protein extract. In one embodiment of a carbon adsorption step, the protein extract is then sent through a food-grade granular charcoal-filled annular basket column (<5% w/w charcoal-to-protein extract ratio) at 4 to 8° C.

In some embodiments, following extraction and optionally carbon adsorption, the clarified protein extract is acidified with a food-safe acidic solution to reach its isoelectric point under chilled conditions (e.g., 2 to 8° C.). Under this condition, the target protein precipitates and becomes separable from the aqueous solution. In some embodiments, the pH of the aqueous solution is adjusted to approximately the isoelectric point of at least one of the one or more globulin-type proteins in the protein-rich fraction, for example, mung bean 8S/beta conglycinin. In some embodiments, the pH is adjusted from an aqueous solution comprising the protein extract which has an initial pH of about 5.0-10.0 prior to the adjusting step. In some embodiments, the pH is adjusted to about 5.0 to 6.5. In some embodiments, the pH is adjusted to about 5.2-6.5, 5.3 to 6.5, 5.4 to 6.5, 5.5 to 6.5, or 5.6 to 6.5. In some embodiments, the pH is adjusted to about 5.2-6.0, 5.3 to 6.0, 5.4 to 6.0, 5.5 to 6.0, or 5.6 to 6.0. In certain embodiments, the pH is adjusted to about pH 5.4-5.8. In some embodiments, the pH is adjusted to about 5.2, 5.3, 5.4, 5.5, 5.6, 5.7, 5.8, 5.9, 6.0, 6.1, or 6.2.

In a preferred embodiment of the methods provided herein, for mung bean protein purification, the pH is adjusted, and precipitation of desired mung bean proteins is achieved, to a range of about pH 5.6 to pH 6.0. Without being bound by theory, it is believed that isoelectric precipitation at a range of about pH 5.6 to pH 6.0 yields a superior mung bean protein isolate, with respect to one or more qualities selected from protein yield, protein purity, reduced retention of small molecular weight non-protein species (including mono and disaccharides), reduced retention of oils and lipids, structure building properties such as high gel strength and gel elasticity, superior sensory properties, and selective enrichment of highly functional 8S globulin/beta conglycinin proteins. These unexpectedly superior features of mung bean protein isolates or mung bean protein concentrates prepared by the methods provided herein are described, for example, in Examples 6 and 8 of US Publication No.: US 2017/0238590 A1. As demonstrated by the results described in Example 6 of US2017/0238590 A1, mung bean protein isolates that underwent acid precipitations at a pH range of about pH 5.6 to pH 6.0 demonstrated superior qualities with respect to protein recovery (in comparison to recovery of small molecules), gelation onset temperature, gel strength, gel elasticity, and sensory properties, in comparison to mung bean protein isolates that underwent acid precipitations at a pH below pH 5.6. Mung bean protein isolates that underwent acid precipitations at a pH range of about pH 5.2 to pH 5.8 also demonstrated substantially lower lipid retention when compared to mung bean protein isolates that underwent acid precipitations outside this range.

Suitable food-grade acids to induce protein precipitation include but are not limited to malic, lactic, hydrochloric acid, and citric acid. In a particular embodiment, the precipitation is performed with a 20% food-grade citric acid solution. In other embodiments, the precipitation is performed with a 40% food-grade citric acid solution.

In some embodiments, in addition to the pH adjustment, EDTA, for example, 2 mM of food-grade EDTA, is added to the precipitation solution to inhibit lipid oxidation in order to produce off-flavor compounds.

In alternative embodiments, the precipitation step comprises isoelectric precipitation at pH 5.6 combined with cryo-precipitation (at 1-4° C.), wherein the pH is adjusted to 5.4-5.8.

In another alternative embodiment, low ionic strength precipitation at high flow rates is combined with cryo-precipitation (at 1-4° C.). In some such embodiments, rapid dilution of the filtrate is performed in cold (1-4° C.) 0.3% NaCl at a ratio of 1 volume of supernatant to 3 volumes of cold 0.3% NaCl. Additional resuspension and homogenization steps ensure production of desired protein isolates.

In some embodiments, the precipitated protein slurry is then removed from the pH-adjusted aqueous solution and sent to a solid/liquid separation unit (for example, a one disc-stack centrifuge). In some embodiments of the methods, the separation occurs with the addition of 0.3% (w/w) food-grade sodium chloride, and a protein curd is recovered in the heavy phase. In preferred embodiments the protein curd is washed with 4 volumes of soft water under chilled conditions (2 to 8° C.), removing final residual impurities such as fibrous solids, salts, and carbohydrates.

In some embodiments of the methods, filtration is used as an alternative, or an addition to, acid precipitation. Without being bound by theory, it is believed that while acid precipitation of the protein aids to remove small molecules, alternative methods such as ultra-filtration (UF) are employed to avoid precipitation/protein aggregation events. Thus, in some embodiments, purifying the protein-rich fraction to obtain the mung bean protein isolate or mung bean protein concentrate comprises performing a filtration, microfiltration or ultrafiltration procedure utilizing at least one selective membrane.

The ultrafiltration process utilizes at least one semi-permeable selective membrane that separates a retentate fraction (containing materials that do not pass through the membrane) from a permeate fraction (containing materials that do pass through the membrane). The semi-permeable membrane separates materials (e.g., proteins and other components) based on molecular size. For example, the semi-permeable membrane used in the ultrafiltration processes of the present methods may exclude molecules (i.e., these molecules are retained in the retentate fraction) having a molecular size of 10 kDa or larger. In some embodiments, the semi-permeable membrane may exclude molecules (e.g., pulse proteins) having a molecular size of 25 kDa or larger. In some embodiments, the semi-permeable membrane excludes molecules having a molecular size of 50 kDa or larger. In various embodiments, the semi-permeable membrane used in the ultrafiltration process of the methods discussed herein excludes molecules (e.g., pulse proteins) having a molecular size greater than 5 kDa, 10 kDa, 15 kDa, 20 kDa, 25 kDa, 30 kDa, 35 kDa, 40, kDa, 45 kDa, 50 kDa, 55 kDa, 60 kDa, 65 kDa, 70 kDa, 75 kDa, 80 kDa, 85 kDa, 90 kDa, or 95 kDa. For example, a 10 kDa membrane allows molecules, including pulse proteins, smaller than 10 kDa in size to pass through the membrane into the permeate fraction, while molecules, including pulse proteins, equal to or larger than 10 kDa are retained in the retentate fraction.

In some embodiments, the washed protein curd solution resulting from acid precipitation and separation is pasteurized in a high temperature/short time pasteurization step to kill any pathogenic bacteria present in the solution. In a particular embodiment, pasteurization is performed at 74° C. for 20 to 23 seconds. In particular embodiments where a dry isolate is desired, the pasteurized solution is passed through a spray dryer to remove any residual water content. The typical spray drying conditions include an inlet temperature of 170° C. and an outlet temperature of 70° C. The final dried protein isolate powder typically has less than 5% moisture content. In some embodiments of the methods described herein, the pasteurization is omitted, to maintain broader functionality of the protein isolate.

The following non-limiting methods are provided to further illustrate the embodiments of the invention disclosed herein. It should be appreciated by those of skill in the art that the techniques disclosed in the examples that follow represent approaches that have been found to function well in the practice of several embodiments of the invention, and thus be considered to constitute examples of modes for its practice. However, those of skill in the art should, in light of the present disclosure, appreciate that many changes can be made in the specific embodiments that are disclosed and still obtain a like or similar result without departing from the spirit and the scope of the invention.

### EXAMPLES

### Example 1: Culturing Cells

Cells are daughter cell lines derived from the commercially available chicken cell line UMNSAH/DF1 (C1F is the Applicant's internal designation of the cells), deposited at American Type Culture Collection (ATCC, Manassas, Va., USA) on Oct. 11, 1996.

Media formulation is a basal media (DMEM/F12) comprising amino acids, vitamins, inorganic salts and other components supplemented with FBS or BCS (bovine calf serum).

### Creation of Master Working Cell Banks (MCWB)

A single vial of cells was retrieved from the C1F master cell bank (MCB) to establish C1F MWCB. Briefly, a C1F MCB cryovial was removed from the liquid nitrogen storage and immediately placed into a 37° C. water bath. The cell suspension was quickly thawed by gently swirling the vial. C1F cell suspension was gradually transferred into 15 mL conical tubes containing 10 mL of pre-warmed culture media in a laminar flow hood. The resultant diluted C1F cell suspension was centrifuged for 5 min at 300×g. The supernatant was aseptically aspirated without disturbing the cell pellet. C1F cells were gently resuspended in culture media and transferred into a 250 mL spin culture flask with a final working volume of 50 mL. Cell density and viability post-thawing were determined to monitor C1F health and for quality control of the established MCB.

C1F cells were cultured under agitation at 125 rpm for a total of 9 days and four steps of scale-up. First, the cells were cultured for 2 days at 37° C. in a humidified incubator with 5% CO₂. The culture was then centrifuged at 300×g for 5 min. Culture supernatant was decanted and C1F cell pellet was resuspended in fresh media and seeded in a final volume of 130 mL in a 500 mL shaking flask. Second, C1F cells were cultured under agitation at 125 rpm for additional 2 days at 37° C. in a humidified incubator with 5% CO₂. The culture was then centrifuged again at 300×g for 5 min; the cell pellet was resuspended in fresh media to a final volume of 340 mL of media in a 1 L shaking flask. Finally, after two days of culture, cell culture was collected and centrifuged at 300×g for 5 min; C1F cell pellet resuspended in fresh media for a final working volume of 880 mL in a 2 L shaking flask. C1F cells were cultured for 2 days under the same conditions, centrifuged at 300×g for 5 min; and the cell pellet was resuspended in fresh media to a final volume of 2.3 L in a 5 L shaking flask. C1F cell culture was placed for 1 additional day under agitation in a humidified incubator with 5% CO₂ and harvested for creation of MWCB.

C1F cells in the final expansion culture were collected and centrifuged at 300×g for 5 min. Cells were resuspended in lower volume of culture media and concentrated C1F cells were sampled and counted using semi-automated cell counting system (Vi-Cell). C1F cells went through another centrifugation cycle of 300×g for 5 min and were resuspended in cryopreservation media (with 10% DMSO) in a range of 20-25 million cell/mL. Cells were frozen in bar-coded cryovials at a rate of −1° C./min from 4° C. to −80° C. during a 16 to 24-hour period in isopropanol chambers. Cells were then transferred and stored in a vapor phase liquid nitrogen storage system (Taylor Wharton (<−175° C.)). Vial content and banked storage position were recorded in a controlled database.

CGMP chain of custody documentation (vial identity confirmation) was utilized to ensure the appropriate vial(s) are retrieved from the MWCB for cell bank release testing and cultured meat production.

### Example 2: Cultured Chicken Production

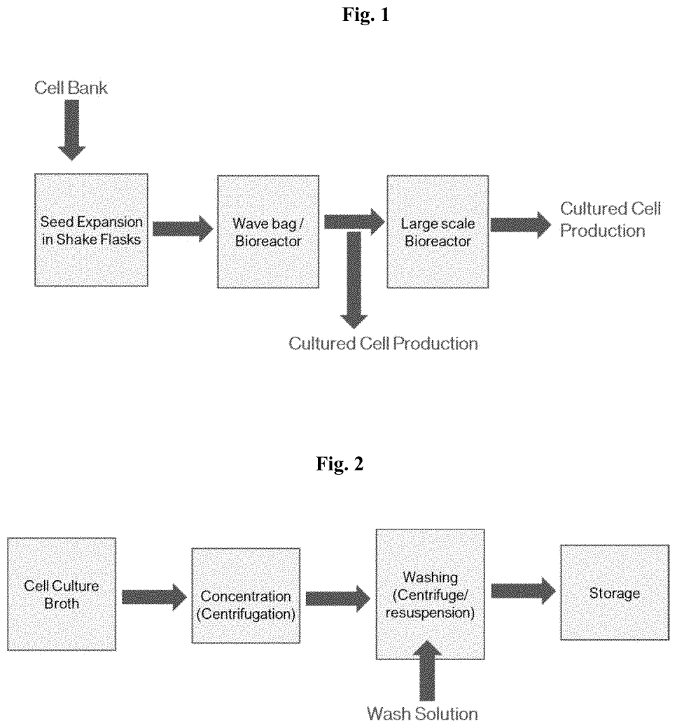

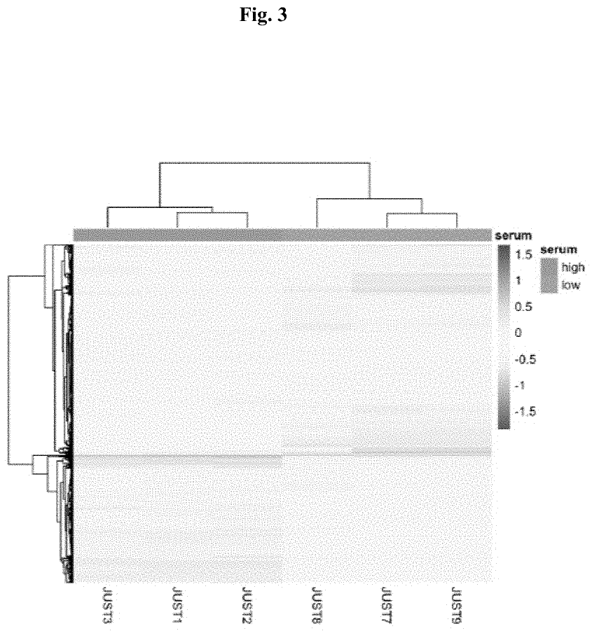

Single-use disposable systems are used for seed expansion and cell growth in the exemplary manufacturing process. The disposal systems with long contact time with the culture media include shake flasks, Wave Bags, media hold bags and stirred tank bioreactor bags for the large-scale 500 L bioreactors. FIG. 1 depicts a process diagram for cell culturing avian fibroblast cells. FIG. 2 depicts a process diagram for harvesting cells.

Seed expansion begins by thawing vials of cells from the MWCB and are cultured in a 500 mL shake flask with 100 mL of working volume. DMEM/F12 with 5% FBS is used in seed expansion. The culture is then split 1:3 to 1:6 and seeded into 1 L shaking flask with a working volume of 300 mL.

The scale-up culture of C1F cells in large shake flasks proceed with a 1:3 to 1:6 split ratio to 900 mL in a 3 L flask followed by 900 mL culture split to 2.7 L in a 5 L flask. Finally, the 2.7 L culture in 5 L flasks is further split in to three 2.7 L flasks using 1:3 split ratios for the transfer into the Wave Bag.

### Cell Culture in Wave Bag

Culture from the three 5 L shake flask (2.7 L culture) is used to inoculate a Wave Bag (total volume of 50 L with a maximum working volume of 25 L) under aseptic conditions, following the 1:3 split ratio previously indicated for the shaking flask cultures (8.1 L of cell suspension+15.9 L of fresh media). Low serum containing media (DMEM/F12+1.25% FBS) is used for the cell growth in the production system (Wave Bag or 500 L bioreactor). 5% serum is used for the cell growth in the Wave Bag if it is used as seed for the 500 L bioreactor.

C1F culture in Wave Bag is either harvested for production or used to inoculate a 500 L bioreactor.

### Culture in 500 L Bioreactor

The contents of the Wave Bag (25 L) are aseptically transferred to a large-scale bioreactor (total volume of 700 L with maximum working volume of 500 L) with 100 L of initial culture media (with a 1:3 to 1:6 split ratio to a total volume of 125 L).

After 3 days (+/−0.5 days) of culture, the media volume is increased to 500 L by the addition of 375 L new culture media and continued for an additional 3 days (+/−0.5 days). Cultures are sampled regularly to determine cell number and viability. Bioreactor culture is monitored off-line for pH, lactate, glucose, glutamine and glutamate levels.

### Concentration and Recovery

The cell culture broth is concentrated (25-100 fold) using a vertical axis flow through decanter centrifuge. The method for cell separation could include centrifugation, filtration, flocculation and combination thereof. The speed of the centrifuge is 500-1000 rcf with a flow rate per bowl size of 0.4-1.2 min⁻¹. The concentrated cell culture slurry is collected and moved to the next stage of washing process.

### Washing the Cells

The carryover of media components in the cultured meat is alleviated by efficiently washing the cell pellet after centrifugation. Specifically, the cell pellet obtained after centrifugation of the spent medium at the end of the cell culture is washed twice sequentially via a resuspension & centrifugation process using five-fold (w/v) 0.45% NaCl solution. By washing, the effective reduction of the media component carryover in the cultured meat is at least 25-fold. Except for glucose, glutamine & sodium, the carryover of the media components is empirically estimated to be very low, <10 ppm based on the 25-fold dilution at the end of washing. Glucose and glutamine are consumed as carbon/nitrogen sources during the cell culture.

The efficiency of washing is tracked by measuring the retained amount of Pluronic F-68 in the second wash solution. The initial concentration of the Pluronic F-68 in the growth media is 0.1% w/v (1000 mg/L). The Pluronic F-68 concentration in the second wash solution was not detectable (<<0.01% w/v) confirming the efficiency of washing in removing the other soluble media components.

Albumin in the wash solutions is detected and quantified using Bovine Albumin ELISA kit (Lifespan Biosciences) with high sensitivity and specificity for bovine serum albumin. In the final wash solution, the albumin concentration was determined to be lower than 10 mg/L and could be in the range of 0-100 ppm (mg/L)

The washed cells (Cultured Chicken) are stored in sealed, food-safe containers at less than or equal −20° C. prior to use for final product formulation.

### Example 3: Testing Safety of Cells for Bacteria and Viruses

Safety and efficacy of the cells is checked at all stages of growth and harvesting of the cells. Cultured C1F cells are evaluated for presence of viral, yeast, and bacterial adventitious agents.

The chicken product is analyzed for the presence of bacteria using protocols from the FDA's Bacteriological Analytical Manual (BAM).

Total Plate Count (TPC) is synonymous with Aerobic Plate Count (APC). As indicated in the US FDA's Bacteriological Analytical Manual (BAM), Chapter 3, the assay is intended to indicate the level of microorganism in a product. Briefly, the method involves appropriate decimal dilutions of the sample and plating onto non-selective media in agar plates. After incubating for approximately 48 hours, the colony forming units (CFUs) are counted and reported as total plate count.

Yeast and mold are analyzed according to methodology outlined in the US FDA Bacteriological Analytical Manual (BAM), Chapter 18. Briefly, the method involves serial dilutions of the sample in 0.1% peptone water and dispensing onto a petri plate that contains nutrients with antibiotics that inhibit microbial growth but facilitate yeast and mold enumeration. Plates are incubated at 25° C. and counted after 5 days. Alternately, yeast and mold are analyzed by using ten-fold serial dilutions of the sample in 0.1% peptone water and dispensing 1 mL onto Petrifilm that contains nutrients with antibiotics that facilitate yeast and mold enumeration. The Petrifilm is incubated for 48 hours incubated at 25 or 28° C. and the results are reported as CFUs.

Escherichia coli and coliform are analyzed according to methodology outlined in the US FDA Bacteriological Analytical Manual (BAM), Chapter 4. The method involves serial decimal dilutions in lauryl sulfate tryptone broth and incubated at 35° C. and checked for gas formation. Next step involves the transfer from gassing tubes (using a 3 mm loop) into BGLB broth and incubated at 35° C. for 48+/−2 hours. The results are reported as MPN (most probable number) coliform bacteria/g.

Streptococcus is analyzed using CMMEF method as described in chapter 9 of BAM. The assay principle is based on the detection of acid formation by Streptococcus and indicated by a color change from purple to yellow. KF Streptococcus agar medium is used with triphenyl tetrazolium chloride (TTC) for selective isolation and enumeration. The culture response is reported as CFUs after incubating aerobically at 35+/−2° C. for 46-48 hours.

Salmonella is analyzed according to methodology outlined in the US FDA Bacteriological Analytical Manual (BAM), Chapter 5. Briefly, the analyte is prepared for isolation of Salmonella then isolated by transferring to selective enrichment media, the plated onto bismuth sulfite (BS) agar, xylose lysine deoxycholate (XLD) agar, and Hektoen enteric (HE) agar. This step is repeated with transfer onto RV medium. Plates are incubated at 35° C. for 24+/−2 hours and examined for presence of colonies that may be Salmonella. Presumptive Salmonella are further tested through various methodology to observe biochemical and serological reactions of Salmonella according to the test/substrate used and result yielded. Due to the small quantity of meat produced in 25 L Wave Bags only 5 grams is tested for Salmonella. Quantities tested from 500 L harvests will be consistent with FDA BAM—Chapter 5.

Cultured chicken was prepared by methods consistent with the examples above. Table 1 indicates that bacteria contamination was negligible when compared to US FDA guidelines.

TABLE 1
Microbiological analysis of Cultured Chicken Meat

| | | | |
|---|---|---|---|
| | Basis | | Representative |
| Parameter | Method | Specification | Example |

Microbiological Analysis

| | | | | | |
|---|---|---|---|---|---|
| Aerobic plate | FDA BAM- | <10,000 | cfu/g | <10 | cfu/g |
| count | Chapter 3 | | | | |
| Coliforms | FDA BAM- | <3 | MPN/g | <3 | MPN/g |
| | Chapter 4 | | | | |
| E. coli | FDA BAM- | <3 | MPN/g | <3 | MPN/g |
| | Chapter 4 | | | | |
| Fecal | CMMEF- | <10 | cfu/g | <10 | cfu/g |
| Streptococcus | Chapter 9 | | | | |

| | | | |
|---|---|---|---|
| Salmonella | FDA BAM- | Not Detected | Not Detected |
| | Chapter 5 | | |

### Mycoplasma Contamination

Cultured C1F cells are considered valid for Mycoplasma detection if a minimum 3% of randomly selected and tested cell vials from each bank are thawed and their culture supernatants provide a negative result using the MycoAlert™Mycoplasma Detection Kit. Following the kit guidelines, the tested samples are classified according to the ratio between Luminescence Reading B and Luminescence Reading A: Ratio<0.9 Negative for Mycoplasma; 0.9<Ratio<1.2 Borderline (required retesting of cells after 24 hours); Ratio>1.2 Mycoplasma contamination.

### Viral Assessment

Viral assessment was performed by analyzing adventitious human and avian virus and bacterial agents through an Infectious Disease Polymerase Chain Reaction (PCR) performed by a third-party (Charles River Research Animal Diagnostic Services)—Human Essential CLEAR Panel; Avian Virus and Bacteria Panel.

C1F from cell banks are considered valid for viral assessment if a minimum of 3% of independent cell vials from the tested bank are thawed and their cell pellets provide a negative result for the full panel of adventitious agents.

Cultured C1F cells are considered approved for absence of adventitious avian and human viral and bacterial agents as the independent cell pellets from each cell bank were negative for the entire human and avian panels.

### Example 4: Cultured Chicken Analysis

The nutritional profile of Cultured Chicken was compared to conventional chicken.

A chemical analysis of Cultured Chicken was performed using moisture, protein content, fat content, ash content, carbohydrate. Moisture content was analyzed using the gravimetric oven drying method using a 10-gram test portion of the Cultured Chicken dried at 105° C. for ≥24 hours in a convection oven. The total crude protein was analyzed based the total nitrogen determined by Dumas combustion method using the LECO FP 628 Nitrogen/Protein Analyzer. The fat content was measured as cumulative fatty acid methyl esters (FAMEs) in ratio to the mass of the starting test portion. A 30 mg dried test portion of cells is subjected to direct transesterification by methanolic hydrochloric acid and FAMEs are separated for analysis by GC-FID by liquid-liquid extraction into heptane. Quantitation was achieved by addition of methyl-10-heptadecenoate as an internal standard added to test samples and the calibration standards. FAMEs identified in this method are constituents of GLC-74X analytical standard purchased from Nuchek Prep Inc., which is a mixture of 15 common saturated and unsaturated FAMEs between methyl octanoate and methyl docosanoate. All other significant peaks in the GC chromatograms were quantitated based on the calibration curve of their closest eluting neighbor. The total ash content was analyzed based on the gravimetric method by using the Milestone Pyro 260 Microwave Oven. The sample was heated to 900° C. for over 50 minutes and then held at 900° C. for 1 hour. The carbohydrate content is calculated by difference from the total of moisture, protein, ash, and fat content.

Table 2 summarizes the percent ash, carbohydrates, protein, and fat of Cultured Chicken compared to conventional boneless chicken breast.

TABLE 2
Nutritional analysis of Cultured Chicken in comparison
with conventional boneless chicken breast.

| | | Dry raw | Dry raw | Dry raw JUST |
|---|---|---|---|---|
| Nutritional | Method | chicken | Cultured | Cultured Chicken |
| package | reference | breast | Chicken | (normalized to 0% ash) |
| Ash | AOAC | 0 | 12 | 0 |
| | 930.30 | | | |
| Carbohydrates | Calculation | 0 | 3 | 3.4 |
| Protein | AOAC | 87.1 | 77.8 | 88.3 |
| | 992.23 | | | |
| Total Fat | AOAC | 8.2 | 8.1 | 9.2 |
| | 996.06 | | | |

Table 3 summarizes the percent saturated, monounsaturated and polyunsaturated fats of Cultured Chicken compared to conventional boneless chicken breast. Fat values are presented as % of specific fat relative to total fat in the sample.

TABLE 3
Summary of the percent saturated, monounsaturated
and polyunsaturated fats of Cultured Chicken.

| Nutritional | Method | Dry raw | Dry raw JUST |
|---|---|---|---|
| package | reference | chicken breast | Cultured Chicken |
| Fat - Saturated | AOAC | 26.1 | 36.8 |
| | 996.06 | | |
| Fat - Monounsaturated | AOAC | 34.1 | 50 |
| | 996.06 | | |
| Fat - Polyunsaturated | AOAC | 17.5 | 7.4 |
| | 996.06 | | |
| Calories | Calculation | 437 | 436 |

The Cultured Chicken is similar to that of conventional chicken when comparing the grams per 100 gram of dry cell paste to dry raw chicken. The overall caloric value of conventional chicken breast and Cultured Chicken is similar. Monounsaturated fats (commonly referred to as the healthy type of fat) represent the type of fat in higher percentage in both conventional and Cultured Chicken (34.1% and 50%, respectively), followed by saturated fats and polyunsaturated fats. Interestingly, the high ash content in Cultured Chicken is due to residual salt, primarily from the 0.45% NaCl washes used to prepare the material, and from the culture medium used to grow the chicken cells. This was also confirmed by the sodium levels in Cultured Chicken (3.6%). When ash is removed from the analysis, protein, fat, and carbohydrate levels are quite consistent between Cultured Chicken and conventional chicken.

### Example 5: Avian Food Product Composition

A representative avian food product composition is described below (by weight percentage) in Table 4.

TABLE 4
Example avian food product composition.

| Ingredient | % by weight |
|---|---|
| Water | 20-40 |
| Cell paste | 25-50 |
| Mung bean | 10-20 |
| Fat | 5-20 |
| transglutaminase | 0.0001-0.0125 |

### Example 6: Chicken Nugget Preparation Recipe

One non-limiting recipe is described below.

First, water was conditioned with disodium phosphate at a concentration between 0.03-0.16%. After the water was conditioned, mung bean protein isolate was added into the conditioned water to prepare a hydrated pulse protein. Next, the cell paste made from C1F cells at a concentration of 25-65% was contacted with the hydrated pulse protein to prepare a cell and protein mixture.

A series of heating steps to the cell and protein mixture was applied. In the first step, the temperature of the cell and protein mixture was ramped up to a temperature between 45-60° C. Seasonings were added at this step. During the second step, the temperature of the cell and protein mixture was maintained at 45-60° C. and transglutaminase was added. The transglutaminase enzymatic reaction was run for 10-20 minutes at a temperature between 45-60° C. During the third step, the transglutaminase enzymatic reaction is stopped by increasing the temperature of the cell and protein mixture to 70° C. to inactivate the enzyme. As discussed herein, transglutaminase covalently is believed to covalently link peptides in the protein isolate together and with peptides present on the cultured cells. During the third step, oil was added at a concentration between 5-20% (v/v).

The cell and protein mixture after treatment of the third step was then cooled to a temperature between 5-15° C.

The cell and protein mixture after the third step was then emulsified in 5-25% fat to create an emulsified mixture that is transferred to a mold. The density and texture of the emulsified mixture was changed by applying a vacuum to the mold. The emulsified mixture was then portioned out into silicone molds/trays. The silicon molds/trays were then baked at 200-275° C. for 5-19 minutes and with 35-75% steam injection.

The baked material was then bagged, flash frozen, or refrigerated. The baked material was then breaded and fried to produce a cultivated avian chicken bite.

The cultivated avian chicken bite was tested by a tasting panel and the panel determined that the cultivated avian cell chicken bite was comparable in taste, texture and mouthfeel to a chicken bite prepared from a farmed animal.

### Example 7: Sequencing Analysis on the Chicken Cells Used for Manufacturing

Sequencing analysis on the chicken cells used for manufacturing was compared to the parental cells to evaluate potential genetic drift induced by the culture conditions.

Briefly, differential gene expression analysis was done using the R program DESeq2_1.20.0, based on the referenced publication. Afterwards, the hierarchical clustering of samples was performed with ClusterProfiler: cluster_2.0.7-1.

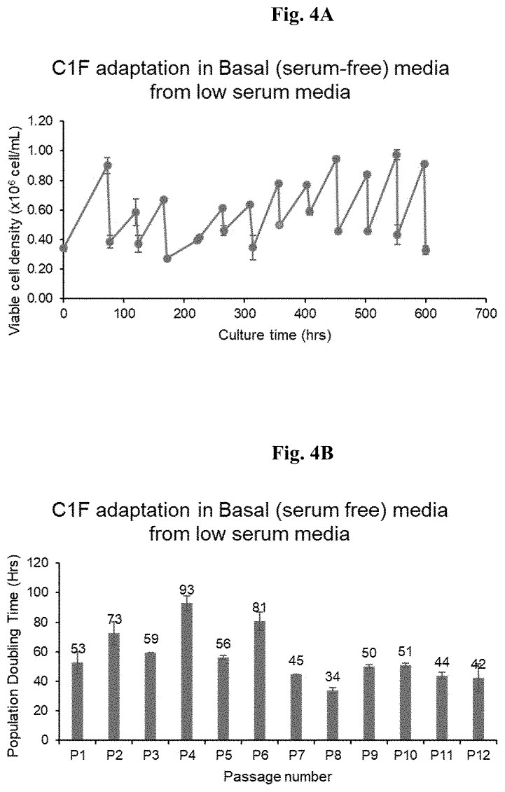

FIG. 3 depicts the clustering analysis performed between three biological replicates of parental chicken cell pools and three biological replicates of chicken cell pools used for manufacturing of Cultured Chicken.

More than 10,400 genes are plotted in FIG. 3, with statistically differently expressed genes selected for p<0.01, and the scale of differently expressed genes being presented as a heatmap.

Samples JUST1-JUST3 were obtained from parental chicken cells cultured in adherent conditions with media supplemented with high (10% v/v) serum concentration. Samples JUST 7-JUST9 were cultured in suspension with media supplemented with low (1.25% v/v) serum concentration. As observed in FIG. 3, samples clustered together within each group, demonstrating homogeneity between biological replicates within each culture condition.

Pathway enrichment was performed using enrichKEGG based on annotations on the Gallus gallus database (GenomelnfoDbData_1.1.0 and Org.Gg.eg.db (Gallus database) v2.1 updated Apr. 9, 2018), to verify if the differently expressed genes were grouped in certain pathways.

Pathways that were influenced include those associated with mechanisms of DNA replication, proteasome, ribosome, apoptosis and steroid biosynthesis. None of up- and down-regulated genes were associated with metabolites, proteins or other toxins harmful for human consumption.

### Example 8: Effect of Reducing Serum Content

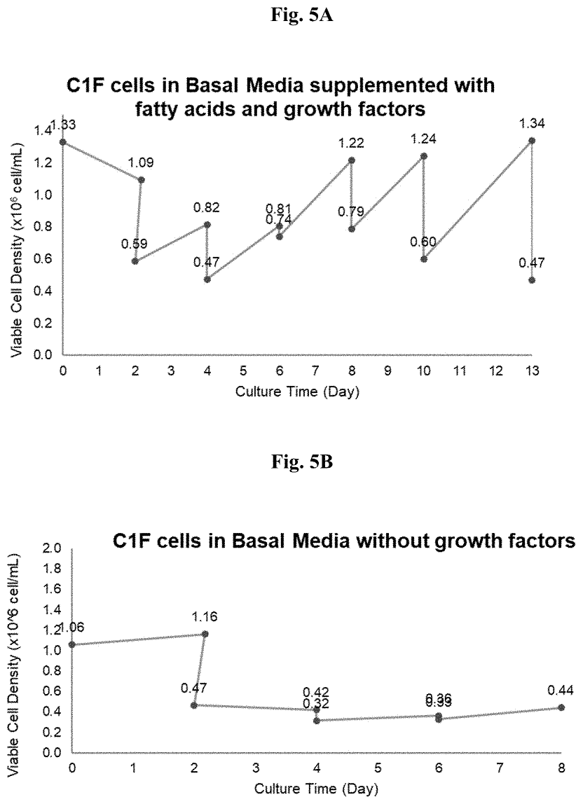

The effect of low serum media on cell viability (FIG. 4A) and population doubling time was analyzed (FIG. 4B). Cells were grown initially at 0.5% (v/v) serum concentration and then lowered to 0% (v/v)—serum-free.

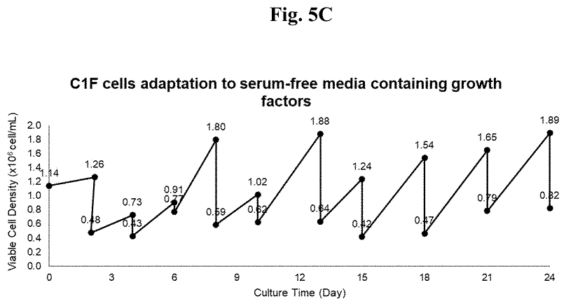

The effect of C1F cell growth in basal media with no serum that was supplemented with fatty acids and growth factors (FIG. 5A), and in basal media with no serum that was supplemented with fatty acids and growth factors (FIG. 5C) were studied and compared to C1F cells grown in basal media with no serum and without growth factors (FIG. 5B). The growth factors used were insulin-like, epidermal-like, and fibroblast-like growth factors at concentrations between 5-200 microgram/mL. FIGS. 5A and 5C used 100 microgram/mL of growth factors during experimentation. Similar effects were observed with growth factors at 50 microgram/mL. The results of which demonstrate that serum free media supplemented with growth factors achieve similar viable cell density as basal media with serum that is supplemented with growth factors.

### Example 9: Adaptation to Serum-Free Conditions

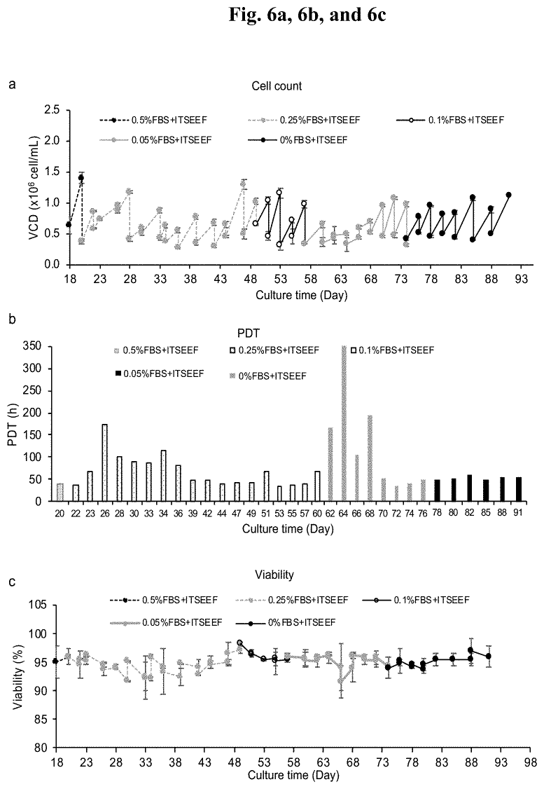

A methodology of gradual adaptation was implemented based on sequential reduction of serum percentage at each step, after assuring successful cell adaptation from the previous step. Cellular adaptation to lower serum concentration is not an immediate process and requires a period of time to get adjusted to the new microenvironment and to acquire a healthy appearance and an obvious growth at each stage of serum reduction. First, we determined the threshold concentration of FBS below which cells in suspension show significant growth arrest. C1F cells maintained in 5% FBS containing media were transferred to 2%, 1% and 0.5% FBS. When FBS concentration was reduced below 1% (v/v), cells showed of reduced growth. In order to adapt cells to low-serum concentration, media containing 1% v/v and 0.5% (v/v) FBS were supplemented with insulin-transferrin-selenium-ethanolamine (ITSE) (ThermoFisher) and growth factors (epidermal growth factor (EGF) and basic fibroblast growth factor (FGF), Peprotech). The use of ITSE, EGF and basic FGF together is referred to as ITSEEF. FIG. 6 discloses viability, population doubling time and population doubling level of cells adapted to grow in serum free media. FIG. 6a shows the viable cell density during the serum weaning process. FIG. 6b shows population doubling time during the serum weaning process. FIG. 6c shows the viability of C1F cells as the cells are transitioned from media containing 0.5% FBS to 0% FBS.

In order to achieve higher cell density in serum-free media, additional chemically defined supplements were tested. As shown in Table 5, vitamins, lipids, and trace elements were screened together with weaning of growth factors and ITSE. In this example, both powder (ThermoFisher, Cat #A42914EK) and liquid (basal media (DMEM/F-12, Cat #11320-033) supplemented with Pluronic-F68 and ITSEEF, so called JUST Basal (JB) media going forth) versions of DMEM/F12 media were used. Liquid DMEM/F12 was used for most of the adaptation study. SFM (SFC-2) with standard osmolarity (around 330 mOsm/Kg) was prepared using a commercially available powdered form of DMEM/F12 while SFM (SFC-4) with low-osmolarity (around 280 mOsm/Kg) was prepared using a custom-made variant of powder DMEM/F12 which did not contain glucose, HEPES buffer, L-glutamine, sodium bicarbonate, and sodium chloride. Missing components of SFC-4 were added separately and osmolarity was adjusted based on different values of sodium chloride addition. In-house RO/DI water was used to prepare DMEM/F12 basal media from powder formulations.

TABLE 5
Composition of the different SFM optimized at different
stages of adaptation to serum-free condition.

| | | SFM Type | | | |
|---|---|---|---|---|---|
| Components | | JB | JB-VLA | SFC-2 | SFC-4 |
| Basal media | DMEM/F12 | X | X | X | X |
| Protein-based | ITSE | X | X | X | X |
| Growth Factors | EGF | X | X | | |
| | Basic FGF | X | X | | |
| Vitamins | 4Vit Mix | | X | X | X |
| Lipids | CDL Mix | | X | X | X |
| Trace Elements | Commercial Trace | | X | X | X |
| | Element Mix | | | | |
| Surfactant | Pluronic | X | X | X | X |

### Serum-Free C1F (SF-C1F) Cell Expansion and Cryopreservation

Based on viable cell density, the split ratio for the expansion of C1F cells was determined, which is typically 1:3 (v/v). C1F cells cultured in serum-free media (SFM) were expanded from 125 mL flask with 50 mL working culture to a final step at 5 L flask with 2.5 L working volume, via multiple incremental subculture steps: 100 mL in 250 mL flasks, 300 mL in 500 mL flasks, 900 mL in 2.8 L flasks. After each cell passage, a new measurement of cell density and viability was done following the same protocol previously described.

SF-C1F cell banks were prepared from actively growing cultures in 5 L shake flask. The volume of C1F cell suspension that held the number of cells desired to bank was centrifuged at 300×g. The supernatant was aseptically decanted or aspirated without disturbing the C1F cell pellet. The cell pellet was gently resuspended in cryopreservation medium. Various in-house and commercially available freezing media were screened to determine the best performer (Table 6). In-house freezing media were prepared by adding FBS and/or DMSO to SFM (SFC-2) media. Commercial cryopreservation media were purchased from BioLife Solutions (CryoStor CS2, CSS, CS10) and PromoCell (Cryo-SFM). SF-C1F cell banks were stored as 20 to 30 million cell aliquots at −185° C. in the vapor phase of a liquid nitrogen freezer. One (1) mL aliquots for in-house cryopreservation media and 2 mL aliquots for commercial freezing media were dispensed into cryogenic storage vials. Cells were frozen in bar-coded cryovials at a rate of −1° C./min from 4° C. to −80° C. during a 16 to 24-hour period in isopropanol chambers. C1F cells were then transferred and stored in a vapor phase liquid nitrogen storage system (Taylor Wharton (<−175° C.)). Vial content and banked storage position were recorded in a controlled database. GMP chain of custody documentation (vial identity confirmation) is utilized to ensure the appropriate vial(s) are retrieved from the cell banks.

Two vials of SF-C1F cell bank were thawed in 37° C. water bath for less than 2 min and resuspended following 10-times dilution using SFM (SFC-2). After centrifugation at 300×g, the supernatant was removed, and the cells were resuspended again in fresh SFM at a density between 0.3-0.6×10⁶ cell/mL. Spin passage was carried out until the cells showed recovery by growing to a viable cell density (VCD) at or above 1.2×10⁶ cell/mL. Upon recovery cells were split passaged following 1:3 ratio. Spin passage was performed by centrifuging the cells at 300×g for 5 min and discarding the supernatant. The cell pellet was then resuspended in fresh medium. In the split passage method, a portion of cell culture was transferred to a new flask containing predetermined amount of fresh media. For a cell split ratio of 1:3, one third of the total volume of the original C1F suspension is transferred to a flask containing two thirds of total volume of fresh culture media. VCD was measured according to the method disclosed in Example 13.

TABLE 6
Cryopreservation media tested for SF-C1F cells.

| Media Type | Name/Components | Vendor | Type |
|---|---|---|---|
| Cryopreservation | 10% FBS + 10% DMSO + | In-house | Serum |
| media | 80% SFM | | containing |
| | 90% FBS + 10% DMSO | In-house | Serum |
| | | | containing |
| | 10% DMSO + 90% SFM | In-house | Serum-free |
| | CryoStor CS2 | BioLife | Serum-free |
| | | Solutions | |
| | CryoStor CS2 | BioLife | Serum-free |
| | | Solutions | |
| | CryoStor CS10 | BioLife | Serum-free |
| | | Solutions | |
| | Cyro-SFM | PromoCell | Serum-free |

For quantification of viable cell density and viability, 1 mL of C1F suspension were collected in an Eppendorf tube and centrifuged at 300×g for 5 min. The supernatant was discarded or used for determination of metabolite concentration. The C1F cell pellet was resuspended in 500 μL of TrypLE Express (Gibco) and incubated for 5-8 min at 37° C. on a shaking platform, followed by an inactivation of enzymatic activity by adding 500 μL of culture media. The total volume (minimum volume of 550 mL per sample) was transferred to sampling cups for the Vi-Celltm XR Cell Viability Analyzer (Beckman Coulter). Cell density and viability was quantified using the Vi-Cell analyzer. Nova Flex bioanalyzer (Nova Biomedical, USA) was used to evaluate values of pH, glucose, glutamine, glutamate, lactate, ammonium, potassium, and sodium. One (1) mL of sample (spent or fresh media) was used for media component and metabolite analysis. The osmolarity of fresh and spent media was measured using OsmoPro osmometer (Advanced Instruments) using 20 μL of sample. Population doubling time (PDT) and Population doubling level (PDL) were calculated according to the following formulas:

PDT=t*log 10(2)/((log 10(n/n0)), where t=culture time, n=final cell number and n0=number of cells seeded.

PDL=3.32[log 10(n/n0)], where n=final cell number and n0=number of cells seeded.

After successful adaptation of the C1F cells to 0.5% FBS, FBS was further reduced in steps to 0.25%, 0.1%, 0.05% and to 0% FBS. C1F cells were successfully grown without FBS in the presence of ITSEEF, however, the cell density and proliferation rate was a little lower than cells grown in 5% FBS containing medium.

### Example 10: Additional Media Components

This example discloses the addition of nutritional components to serum free media to improve cell density and proliferation rate. The media disclosed in Table 5 is referred to as JUST Basal (JB) media. A lipid solution purchased from ThermoFisher (CD-lipid) was previously reported to aid in weaning of FBS in cell culture media. Lipids, especially essential fatty acids and ethanolamine have been shown to support increased growth of cells, including fibroblasts. They store energy and act as constituents of the cellular membrane; they also aid in signaling and transport. Supplementation of chemically-defined vitamins and lipids improved the VCD of serum-free C1F cells from about 0.8-1.0×10⁶ cell/mL to 1.5×10⁶ cell/mL. VCD was measured according to the method disclosed in Example 13. Next, we added trace elements to increase VCD and proliferation rate. For instance, selenium is known to help detoxify free radicals as a cofactor for glutathione (GSH) synthetase. Other trace elements like copper, zinc and tricarboxylic acid are necessary albeit in small quantities for cell growth and proliferation. The micronutrients are also essential for the functionality and maintenance of certain enzymes. Trace elements A, B and C purchased from Corning were tested. Trace A mixture contains defined concentration of CuSO4, ZnSO4, Na-selenite, and ferric citrate. When cultured with Trace A (JB-VLA), C1F cells were able to achieve a VCD of ˜2×10⁶ cell/mL or higher over time. Interestingly Trace B and C had no observable effects on C1F chicken cells growth in SFM. VCD was measured according to the method disclosed in Example 13.

### Example 11: Reduction of Growth Factors

This example discloses the reduction of growth factors in SFM. C1F cells were cultivated as disclosed in Example 10 but were adapted to minimize the addition of growth factors by slowly reducing the amount of growth factors added to the media. Over time, C1F cells grew successfully at similar VCD and proliferation rates as disclosed in Example 10 in media that did not contain EGF and FGF.

Experiments to reduce ITSE were successful in reducing the amount of ITSE supplementation by 10-fold without compromising the growth and proliferation of chicken cells in SFM. VCD was measured according to the method disclosed in Example 13.

### Example 12: Large Scale Manufacturing of Avian Cells

Multiple large scale manufacturing of C1F cells in single use and stainless-steel bioreactors at scales of up to 1,000 L using serum-free (C1F-SFM) and serum-containing media (C1F-SCM) were performed. The serum-free and serum-containing media are described herein.

As the size of the fermentation vessel increases, high pressure, mixing time, nutrient flow, lower O₂ levels and buildup of CO₂, and shear act to inhibit or prevent growth of cells or lysis of the cells. As the size of the fermentation vessel increases in height, the pressure at the bottom of the vessel can be extremely high, leading to lysis of cells. It is a surprising and unexpected result that avian cells could be cultivated in large fermenters. Avian cells do not have a protective cell wall that protects the cell from high pressures.

Single use wavebag bioreactors were used in batch cell culture mode or perfusion mode.

For the batch cell culture mode, culture from 5 L shake flasks was used to inoculate a 50 L rocking motion wavebag under aseptic conditions, to obtain a desired split ratio. After a desired cell density was achieved, the additional media was added to the wavebag to achieve a desired split ratio. At this point the total culture volume was 50 L. Upon completion of the cultivation of the 50 L wavebag, the entire contents of two wavebag batch cultures were used to inoculate 200 L stainless steel bioreactor.

For the perfusion wavebag cultures used to generate inoculum for 500 L bioreactors, a single 50 L wavebag bioreactor was inoculated with culture from 5 L shake flasks and fresh media was added to achieve a desired split ratio. Following inoculation, the cell culture was allowed to grow for one day, before the perfusion process was initiated on day 1. Perfusion was continued for a predetermined amount of time and on the last day of the perfusion, the cell culture was transferred and used to inoculate the 500 L single use bioreactor following desired split ratio.

Multiple 200 L and 1,000 L stainless steel bioreactor cultivations were performed to manufacture cultured chicken cells. The contents of the wavebag culture discussed above were transferred to the 200 L bioreactor and culture media was added to the bioreactor to achieve a desired split ratio. During cultivation, the bioreactor culture was monitored off-line for pH, dissolved oxygen, glucose, glutamine, lactate, ammonium, and osmolarity levels. During the cultivation, samples were collected to confirm the absence of microorganisms.

The 1,000 L stainless steel and the 500 L single-use bioreactors may also be run in a draw and fill method. By this process, a desired amount of a bioreactor culture, for example 750 L of a 1,000 L bioreactor or 375 L of a 500 L bioreactor of culture are harvested into an interim storage container (single use BioBag) and fresh media is immediately added to the remaining culture, returning total volume to 1,000 L or 500 L. Concurrent with the refill operation, the collected cell culture is concentrated for harvesting purposes. Once the bioreactor has been refilled to its desired volume, cultivation was continued to achieve a desired cell density. The draw and fill procedure may be performed multiple times culminating with a final harvest collecting the full culture volume.

Cell harvest is defined as separation and collection of cells from growth media/liquid. Typically, the harvest is performed by centrifugation and washing of residual media components. The cells can be washed with any wash solution, typically water containing 0.45% (w/v) NaCl. The product of harvest, cultured chicken, is also termed “cell paste” which means wet cell pellet generated after centrifugation and washing.

Cell densities far exceeding 2 million cells/mL were routinely obtained.

### Example 13: Reducing Lactate Production

During cell growth, metabolite (e.g. lactate, ammonia, amino acid intermediates) accumulation have been shown to be detrimental to cell growth and productivity at certain concentrations (Claudia Altamirano et al., 2006; Freund & Croughan, 2018; Lao & Toth, 1997; Pereira et al., 2018). In a fed-batch process the accumulation of lactate causes a decrease in culture pH requiring the addition of alkali to maintain pH at setpoint or physiological range. Negatively, the addition of alkali causes an increase in the osmolality of the media and it has been shown that higher osmolality levels strongly inhibit the growth and protein production of most cell lines (Christoph Kuper et al., 2007; Kiehl et al., 2011; McNeil et al., 1999).

The major route of lactate accumulation is the interconversion of pyruvate to lactate which is catalyzed by lactate dehydrogenase (LDH). In mammalian cells, studies have shown that LDH exist either as homo- or hetero-tetramers with a subunit A or B, encoded by LDHA or LDHB respectively (Urbańska & Orzechowski, 2019). Moreover, it has been shown that LDHA catalyzes the forward reaction (pyruvate to lactate) and LDHB catalyzes the backward reaction (lactate to pyruvate). LDHA play a key role in the Warburg effect that occurs in cell lines that do not drive the breakdown of pyruvate through the citric acid cycle, producing lactate from pyruvate even in the presence of oxygen

Oxamate, an analogue of pyruvate, is a strong competitive LDHA inhibitor halting the Warburg effect by channeling much of the breakdown of glucose through the tricarboxylic acid (TCA) cycle—a much more energy efficient process (Wang et al., 2019). However, the use of this molecule inhibits cell proliferation which is a key factor at the earlier stage of production for most industrial mammalian cell lines (Kim et al., 2019; Wang et al., 2019).

C1F cells were cultivated in suspension culture supplemented with 1.25% bovine serum. Different concentrations of sodium oxamate were tested: 1, 3, 5, 10, 30, 60, 100, and 200 mM, and production of lactate, glucose consumption, cell growth rates and cell density were measured.

The specific rates were calculated using daily viable cell concentration and metabolite concentrations for the duration of the cell culture. Specific net growth rates (μ_{N}) were calculated as a change in VCD over a time interval t₁ to t₂ using equation (1):

$${μ}_{N} = \frac{In [ \frac{V C D 2}{V C D 1} ]}{t 1 - t 1} \qquad ( Equation 1 )$$

Specific glucose consumption rate (qGluc) or specific lactate production rate (qLac) were determined using equation (2), where P is glucose or lactate concentration:

$$qGluc or qLac = {μ}_{N} ( \frac{P 2 - P 1}{VCD 2 - VC D 1} ) \qquad ( Equation 2 )$$

Viable cell density (VCD) and viability were determined by the trypan blue exclusion method using the Vi-cell I'm (Beckman Coulter) from 1 mL daily samples taken from shake flask cell culture. Gas and pH values including metabolite (glucose, lactate glutamine, glutamate, ammonium) concentrations were measured using the Bioprofile Flex analyzer (Nova Biomedical). Osmolality was measured using the OsmoPro Multi-Sample Micro-Osmometer (Advanced Instruments) which employs the freezing point technology.

C1F-SCM cells treated with different concentrations of sodium oxamate (1,3,5 and 10 mM), including untreated control cells, were cultured in a batch mode using duplicate shake flasks for 3 days. We observed a 28% (p<0.05) reduction in lactate production for 10 mM oxamate-treated cells and appreciable decrease in lactate production in cells treated with other concentrations of oxamate tested on day 2 compared to the control condition. In addition, we observed a concentration-dependent effect of oxamate on lactate production and glucose consumption by day 2 in oxamate-treated cells, with an increase in oxamate concentration leading to reduced lactate production. Other control parameters were within acceptable physiological ranges.

When the experiment was repeated using higher concentrations of sodium oxamate (10, 15, 20 and 30 mM) of sodium oxamate in a 3-day batch cell culture, lactate production was decreased in a concentration-dependent manner and lactate production was decreased by about 52%.

We went ahead to further increase the concentration (30, 60, 100 and 200 mM) of oxamate for a 3-day batch culture. As expected, a concentration-dependent decrease in lactate production in cells treated with oxamate was observed.

To further examine if the effects of oxamate remained similar in cell culture with metabolic by-products already present, we passaged cells treated with 30 mM oxamate using a 1:3 (v/v) split with fresh media (⅓ of the volume of spent media and ⅔ of fresh media). Having been used in culture for several days, carried over media invariably contains residual amounts (carryover) of metabolites resultant from cell consumption.

C1F cells treated with 30 mM of oxamate showed a continuous linear proliferation up to day 5 of culture, peaking at 2.79×10⁶ cells/mL. Control cultures peaked at day 3 of culture, at 1.64×10⁶ cells/mL and lagged after that. Hence, oxamate-treated culture showed a significant increase in maximum viable cell density of ˜44%. Even though the oxamate-treated cells exhibited a higher cell density by day 5, these C1F-SCM cells still had ˜23% reduction in cumulative lactate production compared to the control group. In addition, oxamate-treated C1F cells showed a decreased specific glucose consumption (qGluc). Cell viability and osmolality of the media were not compromised by the supplementation with oxamate. Ammonium accumulation spiked out for oxamate-treated cultures between days 3 and 5 of culture relative to the non-oxamate-treated control. The pH of the media ended up around 7.0 for the control cultures and 7.2 for oxamate-treated C1F-SCM cells.

The increase in cell density of oxamate treated cells is surprising and unexpected. Studies conducted using oxamate on cancer cells show inhibition of cell proliferation. The inhibitory effect of oxamate on cell proliferation may be due to the dependency of cancer cells on the glycolytic pathway as a source of energy as it represents a faster route for ATP generation than via the TCA cycle (Kim et al., 2019; Lu et al., 2014).

### Example 13: Alternative Sugars

We evaluated the impact of alternative sugars (mannose, fructose and galactose) on the growth and metabolism of an in-house C1F chicken cells grown in suspension cultures containing 1.25% FBS. Specific net growth rate (ρ_{N}) and Specific glucose consumption rate (qGluc) or specific lactate production rate (qLac) were calculated according to equation 1 or 2 as disclosed in Example 12.

Suspension cultures as described herein were cultivated using 3 g/L of the respective sugars were added from day 0 and cultured in a batch mode up to day 3. On day 3 after sampling, an additional 3 g/L of each sugar was added to the respective flasks. By day 3, at the peak cell density, flasks that used glucose as carbon source had the highest viable cell density (˜2.805×10⁶ cells/mL), followed by flasks with mannose (˜2.40×10⁶ cells/mL), then fructose (1.935×10⁶ cells/mL) and lastly galactose (0.915×10⁶ cells/mL).

Though mannose-fed flasks had a lower overall lactate production by day 2, when normalized to day 3 VCD, lactate produced showed a slight increase by 1.7% compared to cells cultured with glucose.

Since we observed that the C1F cells could utilize fructose as a carbon source, we evaluated the effect of different starting concentration of fructose. In one experiment, 6 g/L of fructose was added to one set of duplicate flasks from day 0, and 3 g/L of fructose added each to another set of duplicate flasks from day 0. In the flasks starting with 3 g/L of fructose, an additional 3 g/L of fructose was added on day 1 of one of the duplicates and day 2 of the other duplicate. Overall, the flasks showed similar cell density and growth rate profiles by day 2, though flasks cultured with 6 g/L of fructose from day 0 showed a slight increase in lactate accumulation from day 1 to 36% higher by day 3.

We next evaluated the effect of combining glucose, mannose and fructose on growth and lactate production. Using a design-of-experiment (DOE) approach, 17 batch shake flask runs were carried out evaluating varying combinations of concentrations of glucose, mannose and fructose as energy sources for suspension chicken C1F cells. The experimental design used included 3 factors (glucose, mannose and fructose) and 4 levels (0, 0.5, 1.5 and 3.0 g/L). By day 3, cells with the base carbon sources 3.0 glucose/0.5 fructose/0.5 mannose had the highest viable cell density (VCD) of 3.8×10⁶ cell/mL, followed by 3.0 glucose/0.0 fructose/3.0 mannose and 3.0 glucose/3.0 fructose/3.0 mannose. In addition, the VCD of 3.0 glucose/3.0 fructose/3.0 mannose flasks increased from 3.54×10⁶ cell/mL to 3.78×10⁶ cell/mL by day 4. Interestingly, the above-mentioned flasks showed a similar lactate profile to the control flasks

To maximize VCD, DOE analysis showed the presence of glucose to be very significant (p value=0.001). This was followed by the presence of mannose. In addition, the DOE analysis showed that to maximize VCD with minimal lactate, glucose and mannose combinations needed to be optimized. Moreover, fructose combinations showed lowest lactate accumulation levels and lower VCDs. The cultures with lowest amount of glucose (or no glucose or low/no mannose) performed poorly compared to those with more glucose and certain amount of mannose. Meanwhile, three flasks (3 glucose/1.5 fructose/3 mannose, 3.0 glucose/0.0 fructose/3.0 mannose and 3.0 glucose/3.0 fructose/3.0 mannose) with 3.0 g/L of glucose and at least ≤1.5 g/L mannose showed a slow consumption of glucose.

Since we discovered the importance of the presence glucose in a culture and the additional benefit of mannose in cell culture longevity, we screened different glucose/mannose ratios using DOE. The DOE design used included 2 factors (glucose and mannose) and 4 levels (0.5, 1.5, 3.0 and 4.0 g/L). By day 4 of cell culture we observed that flasks with 3.0 g/L of glucose with additional 1.5-3.0 g/L of mannose exhibited the highest VCDs (˜10-25% increase vs. control) and extended cell culture longevity compared to the control (only 3.0 g/L glucose). Flasks with 3.0 g/L glucose and 1.5 g/L mannose had a VCD of about 3×10⁶ cell/mL where control flasks with 3.0 g/L glucose and no mannose had a VCD of about 2.5×10⁶ cell/mL.

### Example 14: Chicken Skin Prepared from Cultured Avian Cells

A cell-culture based chicken skin product was prepared using cultured avian cells prepared according to the teachings herein. The chicken skin product was prepared by admixing 10%-60% wet cell paste, between 80%-40% water, and between 0.1.%-25% starch, modified starch, or hydrocolloids. The ingredients were mixed together and heated to 65° C. to set the starch. The mixture was then spread thinly onto a sheet and steamed at 160° C.-220° F. until the mixture was gelled, typically about 20 minutes. The gelled product was removed and allowed to cool to room temperature. The cooled, gelled product was broken apart into pieces and dried and brown at 120° C.-160° F. until dry to prepare the cell-culture based chicken skin product. Typically, the drying time was between 4-12 hours.

The cell-culture based chicken skin product had a deep umami flavor profile and mouth feel of chicken skin from a farmed animal. In taste tests, some subjects preferred the taste of the cell-culture based chicken skin product over the skin of farmed chicken.

The embodiments and examples described above are intended to be merely illustrative and non-limiting. Those skilled in the art will recognize or will be able to ascertain using no more than routine experimentation, numerous equivalents of specific compounds, materials and procedures. All such equivalents are considered to be within the scope and are encompassed by the appended claims.

### REFERENCES

Lawler A & Adler J. 2012. Smithsonian Magazine. June. Available at http://www.smithsonianmag.com/history/how-the-chicken-conquered-the-world-87583657/.
USDA Fact Sheets—Poultry Preparation. Focus on: Chicken. Available at http://www.fsis.usda.gov/Fact_Sheets/Chicken_Food_Safety_Focus/index.asp.
Gorman J. 2016. Chickens Weren't Always Dinner for Humans. NY Times. Jan. 18, 2016. Available at www.nytimes.com/2016/01/19/science/chickens-werent-always-dinner-for-humans.html.
English D R, MacInnis R J, Hodge A M, Hopper J L, Haydon A M, Giles G G. 2004. Red meat, chicken, and fish consumption and risk of colorectal cancer. Cancer Epidemiology and Prevention Biomarkers. 13(9):1509-14.
Sinha R, Cross A J, Graubard B I, Leitzmann M F, Schatzkin A. 2009. Meat intake and mortality: a prospective study of over half a million people. Archives of internal medicine. 169(6):562-71; Hu F B, Rimm E B, Stampfer M J, Ascherio A, Spiegelman D, Willett W C. 2000. Prospective study of major dietary patterns and risk of coronary heart disease in men—The American journal of clinical nutrition. 72(4):912-21.
International Agency for Research on Cancer (IARC). 2018. Monographs on the Evaluation of Carcinogenic Risks to Humans. Volume 114. Red Meat and Processed Meat. IARC, Lyon, France.
Physicians Committee for Responsible Medicine (PCRM). 2013. Letter to The Honorable Sanford Bishop, US Congress, dated Mar. 14, 2013.
Altamirano, Claudia, Manes, A., Becerra, S., Cairo, J. J., & Godia, F. (2006). Considerations on the lactate consumption by CHO cells in the presence of galactose. Journal of Biotechnology, 125(4), 547-556. https://doi.org/10.1016/j.jbiotec.2006.03.023
Freund, N. W., & Croughan, M. S. (2018). A simple method to reduce both lactic acid and ammonium production in industrial animal cell culture. In International Journal of Molecular Sciences (Vol. 19, Issue 2). https://doi.org/10.3390/ijms19020385
Lao, M. S., & Toth, D. (1997). Effects of ammonium and lactate on growth and metabolism of a recombinant Chinese hamster ovary cell culture. In Biotechnology Progress (Vol. 13, Issue 5, pp. 688-691). https://doi.org/10.1021/bp9602360
Pereira, S., Kildegaard, H. F., & Andersen, M. R. (2018). Impact of CHO Metabolism on Cell Growth and Protein Production: An Overview of Toxic and Inhibiting Metabolites and Nutrients. Biotechnology Journal, 13(3), 1-13. https://doi.org/10.1002/biot.201700499
Christoph Kuper, Franz-X. Beck, & Wolfgang Neuhofer. (2007). Osmoadaptation of Mammalian Cells—An Orchestrated Network of Protective Genes. Current Genomics, 8(4), 209-218. https://doi.org/10.2174/138920207781386979
Kiehl, T. R., Shen, D., Khattak, S. F., Jian Li, Z., & Sharfstein, S. T. (2011). Observations of cell size dynamics under osmotic stress. Cytometry Part A, 79 A(7), 560-569. https://doi.org/10.1002/cyto.a.21076
McNeil, S. D., Nuccio, M. L., & Hanson, A. D. (1999). Betaines and related osmoprotectants. Targets for metabolic engineering of stress resistance. Plant Physiology, 120(4), 945-949. https://doi.org/10.1104/pp. 120.4.945
Urbańska, K., & Orzechowski, A. (2019). Unappreciated role of LDHA and LDHB to control apoptosis and autophagy in tumor cells. International Journal of Molecular Sciences, 20(9), 1-15. https://doi.org/10.3390/ijms20092085
Wang, Z., Nielsen, P. M., Laustsen, C., & Bertelsen, L. B. (2019). Metabolic consequences of lactate dehydrogenase inhibition by oxamate in hyperglycemic proximal tubular cells. Experimental Cell Research, 378(1), 51-56. https://doi.org/10.1016/j.yexcr.2019.03.001
Kim, E. Y., Chung, T. W., Han, C. W., Park, S. Y., Park, K. H., Jang, S. B., & Ha, K. T. (2019). A Novel Lactate Dehydrogenase Inhibitor, 1-(Phenylseleno)-4-(Trifluoromethyl) Benzene, Suppresses Tumor Growth through Apoptotic Cell Death. Scientific Reports, 9(1), 1-12. https://doi.org/10.1038/s41598-019-40617-3
Lu, Q. Y., Zhang, L., Yee, J. K., Go, V. L. W., & Lee, W. N. (2014). Metabolic consequences of LDHA inhibition by epigallocatechin gallate and oxamate in MIA PaCa-2 pancreatic cancer cells. Metabolomics, 11(1), 71-80. https://doi.org/10.1007/s11306-014-0672-8

1.-60. (canceled)

61. A method of producing an avian cell food product in-vitro, the method comprising the steps of:
a. culturing a population of avian cells in vitro in a growth medium capable of maintaining the avian cells, said growth medium comprising less than 2% fetal bovine serum;
b. recovering the avian cells; and
c. formulating the recovered avian cells into an edible food product.

62. The method of claim 61, wherein the growth medium comprises no fetal bovine serum.

63. The method of claim 62, wherein the avian cells are fibroblasts.

64. The method of claim 62, wherein culturing the population of avian cells is carried out in a suspension culture system, and wherein the cells are grown in a batch, fed-batch, semi continuous (fill and draw) or perfusion culture system.

65. The method of claim 64, said growth medium comprising a growth factor, the growth factor selected from the group consisting of insulin, fibroblast growth factor, and epidermal growth factor.

66. The method of claim 64, wherein the growth factor comprises insulin.

67. The method of claim 64, wherein the growth medium further comprises transferrin.

68. The method of claim 64, wherein the growth medium further comprises selenium.

69. The method of claim 64, wherein the growth medium further comprises ethanolamine.

70. The method of claim 64, wherein the growth medium comprising insulin, transferrin, and selenium.

71. The method of claim 64, wherein the growth medium further comprises a lactate dehydrogenase inhibitor selected from the group consisting of oxamate, galloflavin, gossypol, quinoline 3-dulfonamides, N-hydroxyindole-based inhibitors, and FX11.

72. The method of claim 64, wherein the growth medium further comprises a sugar selected from the group consisting of glucose, galactose, fructose, and mannose.

73. The method of claim 61, wherein the formulating the recovered avian cells into an edible food product comprises the step of admixing a plant protein isolate to the recovered avian cells.

74. The method of claim 73, wherein the pulse protein isolate is a pulse protein isolate.

75. The method of claim 74, wherein the pulse protein isolate is a mung bean protein isolate.

76. The method of claim 73, wherein the formulating the recovered avian cells into an edible food product further comprises contacting a peptide cross-linking enzyme with the avian cell and plant protein isolate admixture.

77. The method of claim 76, wherein the cross-linking enzyme is selected from the group consisting of transglutaminase, sortase, subtilisin, tyrosinase, laccase, peroxidase, and lysyl oxidase.

78. A food product prepared by the method of claim 61.

79. A method of preparing a food product, the food product comprising avian cells cultivated in vitro, the method comprising:
a. conditioning water with phosphates to prepare conditioned water;
b. hydrating a plant protein isolate, with the conditioned water to produce hydrated plant protein;
c. contacting cell paste and hydrated plant protein to produce a cell and protein mixture;
d. heating the cell and protein mixture in steps, wherein the steps comprise at least one of:
i. ramping up the temperature of the cell and protein mixture to a temperature between 40-65° C.;
ii. maintaining the temperature of the cell and protein mixture at a temperature between 40-65° C. for at least 15 minutes;
iii. ramping up the temperature of the cell and protein mixture to a temperature between 60-85° C. to prepare a pre-cooking product;
iv. optionally, cooling the cell and protein mixture to a temperature of a temperature between 5-15° C. to prepare a pre-cooking product;
e. optionally adding an oil at steps (i), (ii), (iii), (iv) or to the pre-cooking product; and
f. optionally, cooking the pre-cooking product to prepare the avian food product.

80. A food product prepared by the method of claim 79.

81. A food product produced from avian fibroblasts cultivated in vitro, the food product comprising:
a. a cell paste, the cell paste content in an amount of at least 25% by weight, and wherein the cell paste is made from avian fibroblast cells cultivated in vitro;
b. a mung bean protein, the mung bean protein content in an amount of at least 15% by weight;
c. a fat, the fat content in an amount of at least 1% by weight; and
d. a water, the water content in an amount of at least 20% by weight.
